SEQUENCE LISTING (10 sequences)

SEQ ID NO: 1 - protein, 1333 aa, Homo sapiens [sequence omitted]

SEQ ID NO: 2 - DNA, 8331 bp, Homo sapiens
ccgccgcccctctccccgcccagaggcgccccgggggcaccatgcaggcgcagcagctgccctacgagtttttcagcgaagagaacgcgcccaagtggcggggactactggtgcctgcgctgaaaaaggtccaggggcaagttcatcctactctcgagtctaatgatgatgctcttcagtatgttgaagaattaattttgcaattattaaatatgctatgccaagctcagccccgaagtgcttcagatgtagaggaacgtgttcaaaaaagtttccctcatccaattgataaatgggcaatagctgatgcccaatcagctattgaaaagaggaagcgaagaaaccctttatctctcccagtagaaaaaattcatcctttattaaaggaggtcctaggttataaaattgaccaccaggtttctgtttacatagtagcagtcttagaatacatttctgcagacattttaaagctggttgggaattatgtaagaaatatacggcattatgaaattacaaaacaagatattaaagtggcaatgtgtgctgacaaggtattgatggatatgtttcatcaagatgtagaagatattaatatattatctttaactgacgaagagccttccacctcaggagaacaaacttactatgatttggtaaaagcatttatggcagaaattcgacaatatataagggaactaaatctaattataaaagtttttagagagccctttgtctccaattcaaaattgttttcagctaatgatgtagaaaatatatttagtcgcatagtagatatacatgaacttagtgtaaagttactgggccatatagaagatacagtagaaatgacagatgaaggcagtccccatccactagtaggaagctgctttgaagacttagcagaggaactggcatttgatccatatgaatcgtatgctcgagatattttgcgacctggttttcatgatcgtttccttagtcagttatcaaagcctggggcagcactttatttgcagtcaataggcgaaggtttcaaagaagctgttcaatatgttttacccaggctgcttctggcccctgtttaccactgtctccattactttgaacttttgaagcagttagaagaaaaaagtgaagatcaagaagacaaggaatgtttaaaacaagcaataacagctttgcttaatgttcagagtggtatggaaaaaatatgttctaaaagtcttgcaaaacgaagactgagtgaatctgcatgtcggttttatagtcagcaaatgaaggggaaacaactagcaatcaagaagatgaacgagattcagaagaatattgatggttgggagggaaaagacattggacagtgttgtaatgaatttataatggaaggaactcttacacgtgtaggagccaaacatgagagacacatatttctctttgatggcttaatgatttgctgtaaatcaaatcatgggcagccaagacttcctggtgctagcaatgcagaatatcgtcttaaagaaaagttttttatgcgaaaggtacaaattaatgataaagatgacaccaatgaatacaagcatgcttttgaaataattttaaaagatgaaaatagtgttatattttctgccaagtcagctgaagagaaaaacaattggatggcagcattgatatctttacagtaccggagtacactggaaaggatgcttgatgtaacaatgctacaggaagagaaagaggagcagatgaggctgcctagtgctgatgtttatagatttgcagagcctgactctgaagagaatattatatttgaagagaacatgcagcccaaggctggaattccaattatcaaagcaggaactgttattaaacttatagagaggcttacgtaccatatgtacgcagatcccaattttgttcggacatttcttacaacatacagatccttttgcaaacctcaagaactactgagtcttataatagaaaggtttgaaattccagagcctgagccaacagaagctgatcgcatagctatagagaatggagatcaacccttgagtgcagaactgaaaagatttagaaaagaatatatacagcctgtgcaactgcgagtattaaatgtatgtcggcactgggtagagcaccacttctatgattttgaaagagatgcatatcttttgcaacgaatggaagaatttattggaacagtaagaggtaaagcaatgaaaaaatgggttgaatccatcactaaaataatccaaaggaaaaaaattgcaagagacaatggaccaggtcataatattacatttcagagttcacctcccacagttgagtggcatataagcagacctgggcacatagagacttttgacctgctcaccttacacccaatagaaattgctcgacaactcactttacttgaatcagatctataccgagctgtacagccatcagaattagttggaagtgtgtggacaaaagaagacaaagaaattaactctcctaatcttctgaaaatgattcgacataccaccaacctcactctgtggtttgagaaatgtattgtagaaactgaaaatttagaagaaagagtagctgtggtgagtcgaattattgagattctacaagtctttcaagagttgaacaactttaatggtgtccttgaggttgtcagtgctatgaattcatcacctgtttacagactagaccacacatttgagcaaataccaagtcgccagaagaaaattttagaagaagctcatgaattgagtgaagatcactataagaaatatttggcaaaactcaggtctattaatccaccatgtgtgcctttctttggaatttatctcactaatatcttgaaaacagaagaaggcaaccctgaggtcctaaaaagacatggaaaagagcttataaactttagcaaaaggaggaaagtagcagaaataacaggagagatccagcagtaccaaaatcagccttactgtttacgagtagaatcagatatcaaaaggttctttgaaaacttgaatccgatgggaaatagcatggagaaggaatttacagattatcttttcaacaaatccctagaaatagaaccacgaaaccctaagcctctcccaagatttccaaaaaaatatagctatcccctaaaatctcctggtgttcgtccatcaaacccaagaccaggtaccatgaggcatcccacacctctgcagcaggagccaaggaaaattagttatagtaggatccctgaaagtgaaacagaaagtacagcatctgcaccaaattctccaagaacaccgttaacacctccgcctgcttctggtgcttccagtaccacagatgtttgcagtgtatttgattccgatcattcgagcccttttcactcaagcaatgataccgtctttatccaagttactctgccccatggcccaagatctgcttctgtatcatctataagtttaaccaaaggcactgatgaagtgcctgtccctcctcctgttcctccacgaagacgaccagaatctgccccagcagaatcttcaccatctaagattatgtctaagcatttggacagtcccccagccattcctcctaggcaacccacatcaaaagcctattcaccacgatattcaatatcagaccggacctctatctcagaccctcctgaaagccctcccttattaccaccacgagaacctgtgaggacacctgatgttttctcaagctcaccactacatctccaacctccccctttgggcaaaaaaagtgaccatggcaatgccttcttcccaaacagcccttccccctttacaccacctcctcctcaaacaccttctcctcacggcacaagaaggcatctgccatcaccaccattgacacaagaagtggaccttcattccattgctgggccgcctgttcctccacgacaaagcacttctcaacatatccctaaactccctccaaaaacttacaaaagggagcacacacacccatccatgcacagagatggaccaccactgttggagaatgcccattcttcctgagttcctctgtactgggatgtatattttcctagccccaaatccattgctggcaatggatgcactgaatgtgccagcactgaggagttaaaatgagaactccaaacactaacgactcttcttcaagatgcagtataagacaatgaattttaacctagatgtaattatacaatggaaatggtattccagtttagaatatggaaagaccgacctagaggaattggacaactgattgcacctgaaaatcataaagggactttttctggccaataggcaggagtcctctttttgtgaagtgatcttttgtgaagtgatcattaaagggatggaaaacacagtctagtgtccagcaggcccacatgacagtttttgtaattcaaattatgcacttttaaaaaaaaaaacttaaacagggatcttaatatcttcctttgttttcctttgctttactcttctactttagaatattttcttaaaaatcactcaaaggactgtgaggaaaggctgtggtacctgaccttgttgaaatcaaggcccggcactgtactacaggcctgtttacagattattacggtgaactgaatgggtaccgaggcttcaccaaagaggtacttttttgttgttgttgttgttttaggaataattgtaccaattttaagagcattccccccacctgtccccacacacccaaacaaaatgtggtggtgttgccttcaaaaaagagaagttttgtgtcattaacatgacagaagaactttttaaaaaaaaataactgtcaactattctatttgcatttaggagactgttcatctatgctagattgtcattttccctccttctcccacagaagtttactggtagtccatgtcatggctcgtagctatccctctaaccataccatggaaatgcaggcacccaatgtgaaaaggagcacttgctgggcatcactgacaccgctcatgttttacacatagttgagtaatcagcatatctagaattatcttgcattgcctaaatcatatgtatatagtgaatgttatataatatacctggcaggtctgttttaatttaattgaataaagatacaaatactttgtttggctggcatatattaaattattatatgaagaaaatggttgattcttatttcttttagttgaaaaacacacaaaaacactaaacataaaagcacgttttgtggtacgccttggtttctagttctggatatatgtattaatcttaaagaatgatctaaagtcacagcttggcatataggaagatacttggttagaataggatgggtatcttttaagcagaactttgtaagtaagttttaagttcctaagcaacaagtatacagtttgttaaggtttatattgaacattctaccttgcttgaggttttctgctttatcaaaactattcataatagaacatctacctgcatcattactatcataatatggattattcataaagcgtatgtactgttcttctgagtctaacaaatactgtgactgaaatgcatgcaccttgttgaagagaaaacattttattaatctccactataatgtctgtgaaatgtgcagtattccatccttagagagtttatctggttcatcagcatctgcatgtatcctctgaataatgaagcaacatttcagcaattctgttaaccacaccattagagacatttttagaagtgctgcaacatggtgggaacctagtactttaataatttcctatgatatttattagagcataaaatgtacatcacgatagtacagtatgtagtcctttaagaccagaaggtatgttaactgcaccatcattaagcagtctatcctattttcttatttatttgttagaattgaatcctttctcaatatctgtaccattcctatgatggtagccattcttctctttataaaatatttcctctttgaaaagcgtcctaaaacattaatttcaaatcccctcttggatgcaaagtgtccctaagaccatccattcaacagtatttatgtgagccaacttactagagccgctacaactaatcatgatagacagtccagaatctccctggactttatactgggccagcctcattgtccctaagatttctctttttatgtctcatggattacttctctttttaattgccccaggcacattccaaatgagaacctggtaaaagtcacattggacccccttcagtatgtcttcggctaatgtgttagccttcaataaggtgataaccccttttcaacctatctagtctataaagaggaaaaattcagatttggagttctaatcctatttctcccaaagggaactattcttctaccccatggttctaggagaccacttcactgtgcaacagttttacatgcattgtggatggaggatatttttattctgaactgttacatttaggtaaaaatattttctgcagtggtaactgattagaaaaatgccaattggatgcccttaggtggaggtgagaaaatggcatccttgccttcttctcaatatgaaacattaactagttgacaaatttatccttgtagtaatgaaaatctatttaatcagggaccagaaatggctgaggagataaatgcatcattacaaaattctgcttttgaatcctggacattacaagggggtaaatgcagcatgactttttgttaaccacattccaaaatgtggaacatttcttttagaaatgaaaatatttcaaggctgatgtattttaagactacacattatcaggaaacatacattgagagttcgcttaattaaaggttgttgggcatcaaattatgtttagtaggttactattctctaacaactcaaggatgctttaatggatctgaatttgacaaagagcatgccacactaatactacagtcaacaacagcccagagaacaattactatgtcagctggaggctatattatgattctaaattcttaaaggtttttttccctccataaatcaaaaattaccttatgtaaaccaaaaattagttggtatttatggtcatgatcttaattctcaagtttagcttaatcttgtatttcattgtttgtcttctaatatgacagcttaaattcagatttttaagtgactcagcaaaataggaggagtgtcccaatttattagtgttgtacatattgaagaaaacctttttgttccttcagatttagaaagaaacagtttaaccatttatttcttggtattctgctgctgtgaataggtttacttattcttttaacatatattgtgtgactacgcaagtataggtcctgttgtattgcattaatctttaccagtaactaaacatcacaaggttaatattggtttggctgaaagaattatgcagtaaagttatttataagggaacatgatgactttattcaatatttttcttctttgaaacatctcattactaacttttaagattatttcataatcccttatacatgagccaatgaaatattttgagctctacttaagaagcatgaagtctatattataaatctaaacaacaaaagcacttgtaacttgtttagtaaattccatgccttattttccatttttgacaccgtaaagtgcattttctgtcgactgtgagcaaatttcccttttgcctttaatgaaaatagtgcattaagtagccaattgctgcagaattataaagcaaattagctaacagtgggtcacattggactaccactcaaagaaacaagatgcataacagatgatggaaggtacatgtggtgtggcttagggagagcgcacctgtgccagatgcatgccactttcagtagctgccaaatgtgcctttaattaagaacatctctaatttttttcttcagatcaagtcagcatttggggtcgggatttgggcagggaactttggggtgcaaatatctggtgaaaccaccagagaatttgtttgtcaaagtgacatgtataattttagttgagacgtgtttgtgtatatatgtgtatttgcctttgtttagtagcacggttatttgctaaaataattgtcaaaaaattgctctgtcggtcggtctttgatgtaagaacaaagtgtggtctatacatctaatatggcctttgtacctaaccatgttcataggtaatctttgtactctgtgtgcagcagtatttggtttgcatttaaagtgaaaataacggctgttatttttctggcattactaagataaactgaaaaataataaagaaaaatgccttacatagcccacaaaaaaaaaaaaaaa

SEQ ID NO: 3 - protein, 1049 aa, Homo sapiens
MQAQQLPYEFFSEENAPKWRGLLVPALKKVQGQVHPTLESNDDALQYVEELILQLLNMLCQAQPRSASDVEERVQKSFPHPIDKWAIADAQSAIEKRKRRNPLSLPVEKIHPLLKEVLGYKIDHQVSVYIVAVLEYISADILKLVGNYVRNIRHYEITKQDIKVAMCADKVLMDMFHQDVEDINILSLTDEEPSTSGEQTYYDLVKAFMAEIRQYIRELNLIIKVFREPFVSNSKLFSANDVENIFSRIVDIHELSVKLLGHIEDTVEMTDEGSPHPLVGSCFEDLAEELAFDPYESYARDILRPGFHDRFLSQLSKPGAALYLQSIGEGFKEAVQYVLPRLLLAPVYHCLHYFELLKQLEEKSEDQEDKECLKQAITALLNVQSGMEKICSKSLAKRRLSESACRFYSQQMKGKQLAIKKMNEIQKNIDGWEGKDIGQCCNEFIMEGTLTRVGAKHERHIFLFDGLMICCKSNHGQPRLPGASNAEYRLKEKFFMRKVQINDKDDTNEYKHAFEIILKDENSVIFSAKSAEEKNNWMAALISLQYRSTLERMLDVTMLQEEKEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 4 - protein, 130 aa, Homo sapiens
MNEIQKNIDGWEGKDIGQCCNEFIMEGTLTRVGAKHERHIFLFDGLMICCKSNHGQPRLPGASNAEYRLKEKFFMRKVQINDKDDTNEYKHAFEIILKDENSVIFSAKSAEEKNNWMAALISLQYRSTLE

SEQ ID NO: 5 - protein, 11 aa, Homo sapiens
ENRIIPHLPAP

SEQ ID NO: 6 - protein, 12 aa, Homo sapiens
KSNHGQPRLPGA

SEQ ID NO: 7 - protein, 12 aa, Homo sapiens
KSNEGQPELPGA

SEQ ID NO: 8 - protein, 4 aa, Homo sapiens
RVQK

SEQ ID NO: 9 - protein, 5 aa, Homo sapiens
KRKRR

SEQ ID NO: 10 - protein, 6 aa, Homo sapiens
KKVQGQ

(19) United States
(12) Patent Application Publication
(10) Pub. No.: US 2012/0301895 A1
(43) Pub. Date: Nov. 29, 2012

(54) METHODS OF MODULATING BINDING OF SON OF SEVENLESS TO PHOSPHATIDIC ACID AND IDENTIFYING COMPOUNDS THAT MODULATE SUCH BINDING

(75) Inventors: Dafna BAR-SAGI, Miller Place, NY (US); Zhao CHEN, Elmhurst, NY (US); Karl SKOWRONEK, Alexandria, VA (US); Kamlesh K. YADAV, New York, NY (US)

(73) Assignees: THE RESEARCH FOUNDATION OF STATE UNIVERSITY OF NEW YORK, Stony Brook, NY (US); NEW YORK UNIVERSITY, New York, NY (US)

(21) Appl. No.: 13/568,878

(22) Filed: Aug. 7, 2012

Related U.S. Application Data
(62) Division of application No. 12/114,914, filed on May 5, 2008, now abandoned.
(60) Provisional application No. 60/916,074, filed on May 4, 2007.

(51) Int. Cl. G01N 33/53

(52) U.S. Cl. 435/7.8

(57) ABSTRACT

The present invention relates to methods of modulating binding of Son of sevenless to phosphatidic acid and identifying compounds that modulate such binding. In particular, the present invention relates to a method of controlling pleckstrin homology domain-dependent membrane recruitment of Son of sevenless or histone folds domain-dependent membrane recruitment of Son of sevenless. Also disclosed are methods of controlling Ras and treating a subject for a condition mediated by Ras. The present invention also relates to a method of identifying compounds potentially effective in treating a condition mediated by Ras.

This application is a divisional of U.S. patent application Ser. No. 12/114,914, filed May 5, 2008, which claims the priority benefit of U.S. Provisional Patent Application Ser. No. 60/916,074, filed on May 4, 2007, each of which is hereby incorporated by reference in its entirety.

The subject matter of this application was made with support from the United States Government under National Institutes of Health grants CA55360 and GM078266. The United States Government has certain rights.

### FIELD OF THE INVENTION

The present invention relates to methods of modulating binding of Son of sevenless to phosphatidic acid and identifying compounds that modulate such binding.

### BACKGROUND OF THE INVENTION

The receptor tyrosine kinase (“RTK”)-Ras signaling axis plays an essential role in the regulation of a wide array of cellular processes, including cell proliferation, differentiation, and survival (Schlessinger et al., “Cell Signaling by Receptor Tyrosine Kinases,” Cell 103:211-225 (2000)). Son of sevenless (“Sos”), the Ras-specific guanine nucleotide exchange factor, couples RTK stimulation to Ras activation by undergoing ligand-stimulated recruitment to the plasma membrane where it promotes the conversion of Ras-GDP to Ras-GTP (Schlessinger et al., “Activation of Ras and Other Signaling Pathways by Receptor Tyrosine Kinases,” Cold Spring Harb. Symp. Quant. Biol. 59:173-179 (1994)). A well established mechanism for the membrane recruitment of Sos involves the formation of a ternary complex with the activated RTK and the adaptor molecule Grb2, through the binding of the Grb2 SH3 domain to a proline-rich sequence in the C-terminus of Sos. In some settings, however, Grb2 binding and the C-terminus of Sos appear to be dispensable for Sos membrane recruitment and Ras activation (Wang et al., “The Grb2 Binding Domain of mSos1 is not Required for Downstream Signal Transduction,” Nat. Genet. 10:294-300 (1995); Karlovich et al., “In Vivo Functional Analysis of the Ras Exchange Factor Son of Sevenless,” Science 268:576-579 (1995)), and genetic and biochemical studies have implicated the Sos N-terminal pleckstrin homology domain (Sos-PH) as the critical determinant (McCollam et al., “Functional Roles for the Pleckstrin and Dbl Homology Regions in the Ras Exchange Factor Son-of-Sevenless,” J. Biol. Chem. 270:15954-15957 (1995); Qian et al., “N Terminus of Sos1 Ras Exchange Factor: Critical Roles for the Dbl and Pleckstrin Homology Domains,” Mol. Cell. Biol. 18:771-778 (1998); Byrne et al., “p21Ras Activation by the Guanine Nucleotide Exchange Factor Sos, Requires the Sos/Grb2 Interaction and a Second Ligand-dependent Signal Involving the Sos N-terminus,” Oncogene 13:2055-2065 (1996)). However, the molecular mechanisms underlying Sos-PH-mediated membrane targeting are not known.

PH domains are well characterized protein- and lipid-interacting modules (Lemmon et al., “Signal-dependent Membrane Targeting by Pleckstrin Homology (PH) Domains,” Biochem. J. 350:1-18 (2000)). It has previously been shown that isolated Sos-PH undergoes ligand-stimulated recruitment to the plasma membrane (Chen et al., “The Role of the PH Domain in the Signal-dependent Membrane Targeting of Sos,” EMBO J. 16:1351-1359 (1997)), implying that this translocation process is specified by sequence elements that lie within the domain itself. It has also been reported that Sos-PH binds phosphatidylinositol 4,5 bisphosphate (“PIP₂”) (Zheng et al., “The Solution Structure of the Pleckstrin Homology Domain of Human SOS1. A Possible Structural Role for the Sequential Association of Diffuse B Cell Lymphoma and Pleckstrin Homology Domains,” J. Biol. Chem. 272:30340-30344 (1997); Kubiseski et al., “High Affinity Binding of the Pleckstrin Homology Domain of mSos1 to Phosphatidylinositol (4,5)-bisphosphate,” J. Biol. Chem. 272:1799-1804 (1997)). However, elimination of the PIP₂ interaction does not interfere with ligand-induced Sos-PH plasma membrane translocation (Chen et al., “The Role of the PH Domain in the Signal-dependent Membrane Targeting of Sos,” EMBO J. 16:1351-1359 (1997)), ruling out PIP₂ binding as the mechanism responsible for membrane targeting.

Phospholipase D (“PLD”), which catalyzes the hydrolysis of phosphatidylcholine to phosphatidic acid (“PA”) and choline, has been implicated in cellular signals that suppress apoptosis and contribute to the survival of cancer cells (Foster et al., “Phospholipase D in Cell Proliferation and Cancer,” Mol. Cancer. Res. 1:789-800 (2003); Foster, “Phospholipase D Survival Signals as a Therapeutic Target in Cancer,” Current Signal Trans. Ther. 1:295-303 (2006)). Elevated PLD activity leads to the elevated expression of Myc (Rodrik et al., “Myc Stabilization in Response to Estrogen and Phospholipase D in MCF-7 Breast Cancer Cells,” FEBS Lett. 580:5647-52 (2006)) and stimulates the activation of mTOR (Fang et al., “Phosphatidic Acid-mediated Mitogenic Activation of mTOR Signaling,” Science 294:1942-5 (2001); Foster, “Regulation of mTOR by Phosphatidic Acid?” Cancer Res. 67:1-4 (2007)), which has been implicated in survival signals in a wide variety of human cancers (Sawyers, “Will mTOR Inhibitors Make it as Cancer Drugs?” Cancer Cell 4:343-8 (2003)). Elevated PLD activity also suppresses the tumor suppressors p53 (Hui et al., “Phospholipase D Elevates the Level of MDM2 and Suppresses DNA Damage-induced Increases in p53,” Mol. Cell. Biol. 24:5677-88 (2004)) and protein phosphatase 2A (Hui et al., “mTOR-dependent Suppression of Protein Phosphatase 2A is Critical for Phospholipase D Survival Signals in Human Breast Cancer Cells,” J. Biol. Chem. 280:35829-35 (2005)). Thus, there is a growing body of evidence implicating PLD in survival signaling, in that many of the downstream targets of PLD have been implicated in the suppression of apoptosis and the survival of cancer cells.

Consistent with the ability of PLD to stimulate signals implicated in cancer cell survival, elevated PLD activity has been observed in several human cancers including breast, kidney, colon, and gastric cancer (Foster, “Phospholipase D Survival Signals as a Therapeutic Target in Cancer,” Current Signal Trans. Ther. 1:295-303 (2006)). In addition, elevated PLD activity has been observed in several human cancer cell lines including those from breast, lung, pancreatic, prostate, and kidney cancers (Chen et al., “Alternative Phospholipase D/mTOR Survival Signal in Human Breast Cancer Cells,” Oncogene 24:672-9 (2005); Zheng et al., “Phospholipase D Couples Survival and Migration Signals in Response to Stress in Human Breast Cancer Cells,” J. Biol. Chem. 281:15862-8 (2006); Gadir et al., “Suppression of TGF-β Signaling by Phospholipase D,” Cell Cycle 6:2840-5 (2007); Shi et al., “Elevated Phospholipase D Activity in Human Cancer Cells with Activating Ras Mutations Provides Survival Signal,” Cancer Lett. 258:268-75 (2007)). More importantly, suppression of PLD activity in cancer cells with elevated PLD activity resulted in apoptosis (Chen et al., “Alternative Phospholipase D/mTOR Survival Signal in Human Breast Cancer Cells,” Oncogene 24:672-9 (2005); Zheng et al., “Phospholipase D Couples Survival and Migration Signals in Response to Stress in Human Breast Cancer Cells,” J. Biol. Chem. 281:15862-8 (2006); Zhong et al., “Phospholipase D Prevents Apoptosis in v-Src-transformed Rat Fibroblasts and MDA-MB-231 Breast Cancer Cells,” Biochem. Biophys. Res. Comm. 302:615-9 (2003); Gadir et al., “Defective TGF-β Signaling Sensitizes Human Cancer Cells to Rapamycin,” Oncogene 27:1055-62 (2007)). Thus, a substantial body of evidence indicates that elevated PLD activity stimulates survival signals in many human cancers.

Activated Ras proteins stimulate increases in PLD activity (Jiang et al., “Involvement of Ral GTPase in v-Src-induced Phospholipase D Activation,” Nature 378:409-12 (1995)). The PLD1 isoform is constitutively associated with RalA (Jiang et al., “Involvement of Ral GTPase in v-Src-induced Phospholipase D Activation,” Nature 378:409-12 (1995); Luo et al., “Ral Interacts Directly with the Arf-responsive PIP2-dependent Phospholipase D1,” Biochem. Biophys. Res. Comm. 235:854-9 (1997)) a downstream target of Ras signaling (Foster et al., “Phospholipase D in Cell Proliferation and Cancer,” Mol. Cancer. Res. 1:789-800 (2003)). Many of the cancer cell lines with elevated PLD activity have activating mutations in Ras (Shi et al., “Elevated Phospholipase D Activity in Human Cancer Cells with Activating Ras Mutations Provides Survival Signal,” Cancer Lett. 258:268-75 (2007)). Thus, a critical target of Ras signaling in human cancers with activating mutations could be PLD. Consistent with this hypothesis, it has been reported that the most critical target of Ras in the transformation of human cells was guanine nucleotide exchange factor for RalA, further implicating PLD in Ras-induced tumorigenesis (Lim et al., “Activation of RalA is Critical for Ras-induced Tumorigenesis of Human Cells,” Cancer Cell 7:533-45 (2005)). Thus, it is possible that the survival of cancer cells with activating mutations to Ras genes may depend on PLD and the downstream targets of PLD signaling which includes mTOR (Foster, “Regulation of mTOR by Phosphatidic acid?” Cancer Res. 67:1-4 (2007)). These studies suggest that suppression of the Ras signals that target RalA could suppress the survival signals mediated by PLD.

The therapeutic targeting of survival signals in cancer cells has appeal because, in principle, suppression of survival signals will result in apoptosis. Honokiol is a natural product isolated from an extract of seed cones from Magnolia grandiflora with antimicrobiol activity (Clark et al., “Atimicrobial Activity of Phenolic Constituents of Magnolia Grandiflora L,” J. Pharm. Sci. 70:951-2 (1981)). Honokiol has more recently been found to have anti-angiogenic properties and blocked tumor growth in mouse xenografts (Bai et al., “Honokiol, a Small Molecular Weight Natural Product, Inhibits Angiogenesis In Vitro and Tumor Growth In Vivo,” J. Biol. Chem. 278:35501-7 (2003)). Honokiol was reported to induce caspase-dependent apoptosis in B-cell chronic lymphocytic leukemia cells (Battle et al., “The Natural Product Honokiol Induces Caspase-dependent Apoptosis in B-cell Chronic Lymphocytic Leukemia (B-CLL) Cells,” Blood 106:690-7 (2005)), and to inhibit the bone metastatic growth of human prostate cancer cells (Shigemura et al., “Honokiol, a Natural Plant Product, Inhibits the Bone Metastatic Growth of Human Prostate Cancer Cells,” Cancer 109:1279-89 (2007)).

The present invention is directed to overcoming limitations in the art.

### SUMMARY OF THE INVENTION

One aspect of the present invention relates to a method of controlling pleckstrin homology domain-dependent membrane recruitment of Son of sevenless or histone folds domain-dependent membrane recruitment of Son of sevenless. This method involves selecting a cell where control of pleckstrin homology domain membrane recruitment of Son of sevenless or histone folds domain-dependent membrane recruitment of Son of sevenless is needed and modulating binding of Son of sevenless to phosphatidic acid in the cell under conditions effective to control pleckstrin homology domain-dependent membrane recruitment of Son of sevenless or histone folds domain-dependent membrane recruitment of Son of sevenless.

Another aspect of the present invention relates to a method of controlling Ras. This method involves selecting a cell where control of Ras is needed and modulating binding of Son of sevenless to phosphatidic acid in the cell under conditions effective to control Ras.

A further aspect of the present invention relates to a method of treating a subject for a condition mediated by Ras. This method involves selecting a subject having a condition mediated by Ras and modulating binding of Son of sevenless to phosphatidic acid in the subject under conditions effective to treat the condition mediated by Ras.

Yet another aspect of the present invention relates to a method of identifying compounds potentially effective in treating a condition mediated by Ras. This method involves providing one or more candidate compounds and contacting each of the candidate compounds with a cell. The effect of the candidate compounds on binding Son of sevenless to phosphatidic acid is evaluated. Candidate compounds which modulate binding of Son of sevenless to phosphatidic acid are identified as compounds potentially effective in treating a condition mediated by Ras.

### BRIEF DESCRIPTION OF THE DRAWINGS

FIG. 1 shows phosphatidic acid binding sites on Son of sevenless protein, including putative PA binding sites I (73-RVQK-76 (SEQ ID NO:8)), II (96-KRKRR-100 (SEQ ID NO:9)), and III (28-KKVQGQ-34 (SEQ ID NO:10)) of the histone folds domain. Also shown are sites H475 and R479 of the PH domain.

FIG. 2 is a schematic illustration showing two cases of Sos localization and Ras-MAPK signaling in cells.

FIG. 3 is a schematic illustration showing a simplified mechanism of action of PLD in bringing about the conversion of phosphatidylcholine to phosphatidic acid.

FIGS. 4A-D show that the PH domain of Sos (Sos-PH) binds to Phosphatidic Acid. FIG. 4A is the domain structure of Sos. HF, Histone folds; DH, Dbl homology; PH, Pleckstrin homology; REM, Ras exchange motif; CDC25, homology region with yeast CDC25; PxxP, Proline-rich Grb2 binding motifs. Sequence alignment of the region exhibiting similarity in Sos-PH (SEQ ID NO:6) and p47^{phox}′-PX (SEQ ID NO:5) is shown in the bracket. The residues found to be critical for the interaction of Sos-PH (see FIG. 4C) with PA are underlined. FIG. 4B shows T7-tagged Sos-PH (0.5 μM) mixed with the indicated concentrations of lipid vesicles comprised of PC:PA (90:10 mole percent) or PC. The vesicles were pelleted by centrifugation and the associated Sos-PH detected by Western blotting with anti-T7 antibody. The results shown are representative of three independent experiments. FIG. 4C shows increasing concentrations of Sos-PH that were incubated with 1 mM PC:PA (circles) or PC (triangles) lipid vesicles. Sos-PH-HR/EE (squares) was incubated with 1 mM PC:PA lipid vesicles under the same conditions as for Sos-PH. The vesicles were pelleted by centrifugation, and the amounts of Sos-PH in the supernatant and pellet determined as described in the Examples. Binding curves were generated using Sigma Plot 10.0, and the apparent dissociation constants derived from the binding curves by fitting the data points to Scatchard binding equations. In FIG. 4D, Sos-PH and Sos-PH-HR/EE (0.5 μM) were incubated with lipid vesicles (1 mM) comprised of PC, PC:PA, or PC:PIP₂ (98:2 mole percent). Binding analyses were performed as described in (FIG. 4B). The results shown are representative of three independent experiments.

FIG. 5 shows a comparison between the binding of Sos-PH to PS-, PA-, or PIP₂-containing lipid vesicles. T7-tagged Sos-PH (0.5 μM) was incubated with 1 mM lipid vesicles comprised of PC:PA (90:10 mole percent), PC:PS (90:20 mole percent), or PC:PIP₂ (98:2 mole percent). The vesicles were pelleted by centrifugation and the associated protein detected by Western blotting with anti-T7 antibody. The results shown are representative of three independent experiments.

FIGS. 6A-D show that the interaction between Sos-PH and PA is essential for serum-induced Sos membrane recruitment and Sos-mediated Ras activation. In FIG. 6A, COS-1 cells were transfected with GFP-tagged constructs of Sos-PH or Sos-PH-HR/EE. The cells were serum-starved and then treated with PA (100 μM) for 20 min. In FIG. 6B, COS-1 cells were transfected with HA-tagged Sos-ΔC or Sos-ΔC-HR/EE. The cells were serum-starved and then stimulated with serum (20%) for 10 min. For FIGS. 6A-B, the fluorescence staining patterns were analyzed through the acquisition of serial optical Z sections. The images shown represent single 0.25 μm optical sections acquired at the mid plane of the cells with the same exposure time. Membrane localization is reflected by the relative increase in fluorescence intensity at the cell periphery (arrowheads). The boxed areas are enlarged on the top and bottom of the corresponding images. The number of cells displaying plasma membrane localization of the protein is expressed as percentage of the total number of cells scored and normalized to the maximal value obtained for each experiment. For each experiment, at least 50% of the cells displayed membrane localization. Results are the mean+/−s.d. of three independent experiments with at least 100 cells scored in each experiment. Scale bars represent 20 μm in all panels. In FIGS. 6C-D, COS-1 cells were co-transfected with HA-tagged Ras and the indicated HA-tagged Sos-ΔC constructs. Cells were serum-starved and then stimulated with EGF (10 nM) for 5 min (FIG. 6C) or with PA (100 μM) for the indicated intervals (FIG. 6D). Ras activation was measured by the RBD pull-down assay as described in the Examples. The results shown are representative of three independent experiments.

FIGS. 7A-B demonstrate that under the experimental conditions used, endogenous Sos does not contribute to the measured EGF-induced Ras activation (FIG. 7A). COS-1 cells were cotransfected with HA-tagged Ras and Sos constructs as indicated. Cells were serum-starved and then stimulated with EGF (10 nM) for 10 min. Ras activation was measured by RBD pull-down. The results shown are representative of three independent experiments. In FIG. 7B, Sos-mediated Ras activation in response to EGF stimulation is shown to not be dependent on Grb2 binding. COS-1 cells were cotransfected with HA-tagged Ras and Sos constructs as indicated (FL, full-length). Cells were serum-starved and then stimulated with EGF (10 nM) for the specified intervals. Ras activation was measured by RBD pull-down. The results shown are representative of three independent experiments.

FIGS. 8A-B show that Sos-mediated Ras activation in response to EGF stimulation requires PA binding (FIG. 8A). COS-1 cells were cotransfected with HA-tagged Ras and Sos constructs as indicated. Cells were serum-starved and then stimulated with EGF (10 nM) for the specified intervals. Ras activation was measured by RBD pull-down. The results shown are representative of three independent experiments. In FIG. 8B, the catalytic activity of PLD2 is shown to be required for the stimulation of Ras activation. COS-1 cells were cotransfected with HA-tagged Ras and wild-type PLD2 or PLD2 mutant that is catalytically defective (PLD2-K758R). The cells were serum-starved and then analyzed for Ras activation by RBD pull-down. The results shown are representative of three independent experiments.

FIGS. 9A-E show that PLD2-mediated signaling is essential for Sos membrane recruitment and Sos-mediated Ras activation. In FIG. 9A, COS-1 cells were co-transfected with GFP-tagged PLD2 and HA-tagged constructs of either Sos-AC or Sos-AC-HR/EE and then serum-starved. The images were captured as in FIGS. 6A-D. Plasma membrane localization is indicated by fluorescent signal at the cell periphery (arrowheads). The number of cells displaying membrane co-localization of the proteins is presented as percentage of total number of cells expressing both proteins. Results are the mean+/−s.d. of three independent experiments with at least 100 cells scored in each experiment. Scale bars represent 5 μm in all panels. In FIG. 9B, COS-1 cells were cotransfected with HA-tagged Ras and the indicated HA-tagged Sos-ΔC and PLD2 constructs. The cells were subsequently serum-starved and Ras activation measured by RBD pull-down. The results shown are representative of three independent experiments. In FIGS. 9C-D, HeLa cells were transfected with the indicated shRNA constructs in the absence or presence of the PLD2 wobble mutant. Following blasticidin selection of transfected cells, the cells were serum-starved and then stimulated with EGF (10 nM) for the indicated intervals (FIG. 9C) or 5 min (FIG. 9D). Ras activation was measured by RBD pull-down assay. The results shown are representative of three independent experiments. In FIG. 9E, HeLa cells were transfected with the indicated shRNA constructs. Following selection, the cells were serum-starved and then stimulated with EGF (10 nM) for the indicated intervals. PLC-γ1 was immunoprecipitated and its phosphorylation analyzed by Western blotting with anti-phosphotyrosine antibody. Results shown are representative of two independent experiments.

FIG. 10 shows that serum-induced membrane recruitment of Sos-PH is dependent on PLD2. COS-1 cells were co-transfected with GFP-tagged Sos-PH and PLD2-shRNA construct co-expressing dsRed, and stimulated with 20% serum for 10 min. The fluorescence staining patterns were analyzed through the acquisition of serial optical Z sections. The images shown represent single 0.25 μm optical sections acquired at the mid plane of the cells. Membrane localization is indicated by fluorescent signal at the cell periphery (arrowheads). The number of cells displaying membrane localization of the proteins is presented as percentage of total number of cells expressing GFP-PH-Sos or GFP-PH-Sos and DsRed-PLD2-shRNA. Results are the mean+/−s.d. of three independent experiments with at least 100 cells scored in each experiment. The scale bar represents 10 μm.

FIGS. 11A-B show that the transforming activity of Ras is potentiated by PLD2. In FIG. 11A, NIH 3T3 cells stably expressing PLD2 were transiently transfected with the indicated H-Ras and Sos-ΔC constructs. After 14 days, the dishes were stained with Giemsa to visualize the foci. Focus forming activity was quantitated by counting the number of foci per culture dish. The data are averages of three culture dishes+/−s.d. and are representative of three independent assays. In FIG. 11B, the expression levels of ectopically expressed proteins were analyzed by Western blotting and the levels of activated Ras determined by RBD pull-down.

FIGS. 12A-C show that stress-induced PLD activity in MDA-MB-231 cells is dependent on Ras and RalA. In FIG. 12A, MDA-MB-231 cells were plated. 24 hr later the cells were placed in fresh media containing either 10% or 0.5% serum. 18 hr later, [³H]-Myristate was added and 4 hr later, BtOH (0.8%) was added for 20 min, at which time the cells were harvested and the extracted membrane lipids were separated by thin layer chromatography to determine the levels of the transphosphatidylation product phosphatidyl-BtOH (PBt). The levels of PLD1, PLD2, and actin were determined using Western blot analysis using the corresponding antibodies. In FIG. 12B, MDA-MB-231 cells were plated and then transiently transfected with an empty vector control or vectors that express either an S17N Ras, a T31N ARF1, a T27N ARF6, or an S28N RalA, mutant 24 hr later. The cells were placed in media containing 0.5% serum 24 hr later and the PLD activity was determined as in FIG. 12A after an additional 24 hr. In FIG. 12C, MDA-MB-231 cells were plated as in FIG. 12A and placed in either 10% or 0.5% serum 24 hr later. At this point the cells were harvested and lysates were prepared and treated with the Ras binding domain of Raf1 (Pierce, Rockford, Ill.) according to the vendor's instruction. Ras-GTP bound to the Raf1 binding domain was recovered and subjected to Western blot analysis using a Pan-Ras antibody supplied with the Ras activation assay kit. 1 mg of protein from whole cell lysates was used for the Ras pull-down assay and 20 μg of whole cell lysates was loaded onto the gel used for the Western blot. Each experiment was repeated at least two times with equivalent results.

FIGS. 13A-B show that honokiol suppresses stress-induced PLD activity in MDA-MB-231 human cancer cells. In FIG. 13A, MDA-MB-231 cells were plated as in FIGS. 12A-C and then shifted to either 10% serum or 0.5% serum overnight as indicated. Honokiol (20 μM) or control ethanol was then added for 4 hr as indicated, at which time the cells were harvested and the levels of PBt were determined as in FIGS. 12A-C. The experiment shown is representative of at least three independent experiments. In FIG. 13B, the effect of honokiol on the activity of PLD isoforms was measured using exogenous substrate assay as described previously (Henage et al., “Kinetic Analysis of a Mammalian Phospholipase D: Allosteric Modulation by Monomeric GTPases, Protein Kinase C, and Polyphosphoinositides,” J. Biol. Chem. 281:3408-17 (2006), which is hereby incorporated by reference in its entirety). Partially purified proteins were incubated in the presence or absence of the indicated concentrations of honokiol for 30 min at 37° C. ARF-1 was included in the PLD1 assay because there is no activity without an Arf protein. The amount of background was determined and subtracted using a bovine serum albumin standard. Error bars represent the standard deviation for triplicate values.

FIGS. 14A-E show that honokiol suppresses Ras activation. In FIG. 14A, MDA-MB-231 cells were plated and then placed in media containing 0.5% media 24 hr later. 24 hr later, the cells were treated with either DMSO or honokiol (20 μM) as indicated for 2 hr. At this point the cells were harvested and lysates were prepared and treated with the immobilized Ras binding domain of Raf1 as in FIGS. 12A-C. Ras-GTP bound to the Raf1 binding domain was recovered and subjected to Western blot analysis using the Pan-Ras antibody. Total Ras in the cell lysates and actin levels were also examined by Western blot analysis. In FIG. 14B, MDA-MB-231 cells were plated and 24 hr later were shifted to media containing 0.5% serum. The cells were then treated with EGF (200 ng/ml) for 10 min. The cells were also treated with honokiol (20 μM) prior to the addition of EGF for the times indicated. The levels of GTP-bound Ras, total Ras, and actin were then determined as in FIG. 14A. In FIG. 14C, Cos1 cells were transfected with plasmids expressing HA-tagged Ras (50 ng) and Sos (500 ng) for 24 hours. The cells were placed in serum free media overnight in the presence of increasing concentration of honokiol. Cells were then collected and Ras activation was determined using the pull-down assay as above. Levels of Ras-GTP, Sos, and Ras were determined by Western blot analysis using an antibody that recognized HA. In FIG. 14D, Cos1 cells were tranfected with the plasmid expressing HA-tagged Ras (50 ng) for 24 hours. The cells were placed in serum free media overnight in the presence and absence of honokiol (50 μM) as indicated. Cells were then stimulated with 100 ng/ml EGF for the indicated times (min) and Ras activation was determined as in FIG. 14A. In FIG. 14E, Cos1 cells were tranfected with plasmids expressing HA-tagged Ras (10 ng) and Sos (100 ng) for 24 hours. The Sos plasmid was not included in the lane at the far right. The cells were placed in serum free media overnight in the presence and absence of honokiol (50 μM) as indicated. Cells were then stimulated with 100 ng/ml EGF for the indicated times (min) and Ras activation was determined as in FIG. 14A. Experiments shown are representative of at least two independent experiments.

FIGS. 15A-B show that honokiol suppresses downstream targets of PLD survival signals. In FIG. 15A, MDA-MB-231 cells were plated in DMEM with 10% serum. 48 hr later the cells were shifted to 0.5% for 16 hr. Honokiol (20 μM) or control ethanol was then added for the indicated times. The cells were then harvested and analyzed for levels of S6K, phosphorylated S6K (P-S6K), 4E-BP1, and P-4E-BP1 by Western blot analysis as described previously (Zheng et al., “Phospholipase D Couples Survival and Migration Signals in Response to Stress in Human Breast Cancer Cells,” J. Biol. Chem. 281:15862-8 (2006), which is hereby incorporated by reference in its entirety). In FIG. 15B, 786-O cells were plated and then shifted to media containing 0.5% serum 24 hr later. 18 hr later, the cells were treated with 1-Bt0H (0.8%), honokiol (Hon) (20 μM), and t-BtOH (0.8%) as indicated. Cell lysates were prepared 4 hr later and examined for HIF2α and actin expression by Western lot analysis. The experiment shown is representative of two independent experiments.

FIG. 16 shows that honokiol induces apoptosis in MDA-MB-231 deprived of serum. MDA-MB-231 cells were plated in DMEM with 10% serum for 48 hr then changed to DMEM with either 10% or 0.5% serum overnight as indicated. Honokiol (20 μM) or control ethanol was then added either at the time of changing media (24 hr time point), or 4 hr prior to harvesting (4 hr time point) as indicated. The cells were then examined for cell viability and PARP cleavage as described previously (Chen et al., “Alternative Phospholipase D/mTOR Survival Signal in Human Breast Cancer Cells,” Oncogene 24:672-9 (2005), which is hereby incorporated by reference in its entirety). The Western blot is representative of at least three independent experiments. The error bars for cell viability represent the standard deviation for triplicate samples from a representative experiment repeated three times.

FIGS. 17A-B show that honokiol suppresses stress-induced PLD activity in T24 bladder and Calu1 lung cancer cells. In FIG. 17A, T24 and Calu1 cells (obtained from the American Type Culture Collection) were plated in DMEM containing 10% serum for 48 hr and then shifted to either 10% serum or 0.5% serum overnight as indicated. Honokiol (20 μM) or control ethanol was then added for 4 hr as indicated. [³H]-Myristate was added with the honokiol. After 4 hr, BtOH (0.7%) was added for 20 min, at which time the cells were harvested and the level of phosphatidyl-BtOH (P-Bt) was determined as in FIGS. 12A-C. The experiments shown are representative of at least two independent experiments. In FIG. 17B, T24 and Calu-1 cells were plated in DMEM with 10% serum for 48 hr then changed to DMEM containing either 10% or 0.5% serum overnight as indicated. Honokiol (20 μM) or control ethanol was then added either at the time of changing media (24 hr), or 4 hr before harvesting as in FIGS. 14A-E, at which time the cells were examined for cell viability or PARD cleavage. The Western blot is representative of at least two independent experiments. The error bars for cell viability represent the standard deviation for triplicate samples from a representative experiment repeated two times.

### DETAILED DESCRIPTION OF THE INVENTION

The present invention is directed to methods which involve modulating binding of Son of sevenless to phosphatidic acid and identifying compounds that modulate such binding.

Son of sevenless (GenBank Accession No. NM 005633) has the amino acid sequence of SEQ ID NO:1, as follows.

```
Met Gln Ala Gln Gln Leu Pro Tyr Glu Phe Phe Ser Glu Glu Asn Ala
1 5 10 15
Pro Lys Trp Arg Gly Leu Leu Val Pro Ala Leu Lys Lys Val Gln Gly
                        20 25 30
Gln Val His Pro Thr Leu Glu Ser Asn Asp Asp Ala Leu Gln Tyr Val
                35 40 45
Glu Glu Leu Ile Leu Gln Leu Leu Asn Met Leu Cys Gln Ala Gln Pro
        50 55 60
Arg Ser Ala Ser Asp Val Glu Glu Arg Val Gln Lys Ser Phe Pro His
65 70 75 80
Pro Ile Asp Lys Trp Ala Ile Ala Asp Ala Gln Ser Ala Ile Glu Lys
                                85 90 95
Arg Lys Arg Arg Asn Pro Leu Ser Leu Pro Val Glu Lys Ile His Pro
                        100 105 110
Leu Leu Lys Glu Val Leu Gly Tyr Lys Ile Asp His Gln Val Ser Val
                115 120 125
Tyr Ile Val Ala Val Leu Glu Tyr Ile Ser Ala Asp Ile Leu Lys Leu
        130 135 140
Val Gly Asn Tyr Val Arg Asn Ile Arg His Tyr Glu Ile Thr Lys Gln
145 150 155 160
Asp Ile Lys Val Ala Met Cys Ala Asp Lys Val Leu Met Asp Met Phe
                                165 170 175
His Gln Asp Val Glu Asp Ile Asn Ile Leu Ser Leu Thr Asp Glu Glu
                        180 185 190
Pro Ser Thr Ser Gly Glu Gln Thr Tyr Tyr Asp Leu Val Lys Ala Phe
                195 200 205
Met Ala Glu Ile Arg Gln Tyr Ile Arg Glu Leu Asn Leu Ile Ile Lys
        210 215 220
Val Phe Arg Glu Pro Phe Val Ser Asn Ser Lys Leu Phe Ser Ala Asn
225 230 235 240
Asp Val Glu Asn Ile Phe Ser Arg Ile Val Asp Ile His Glu Leu Ser
                                245 250 255
Val Lys Leu Leu Gly His Ile Glu Asp Thr Val Glu Met Thr Asp Glu
                        260 265 270
Gly Ser Pro His Pro Leu Val Gly Ser Cys Phe Glu Asp Leu Ala Glu
                275 280 285
Glu Leu Ala Phe Asp Pro Tyr Glu Ser Tyr Ala Arg Asp Ile Leu Arg
        290 295 300
Pro Gly Phe His Asp Arg Phe Leu Ser Gln Leu Ser Lys Pro Gly Ala
305 310 315 320
Ala Leu Tyr Leu Gln Ser Ile Gly Glu Gly Phe Lys Glu Ala Val Gln
                                325 330 335
Tyr Val Leu Pro Arg Leu Leu Leu Ala Pro Val Tyr His Cys Leu His
                        340 345 350
Tyr Phe Glu Leu Leu Lys Gln Leu Glu Glu Lys Ser Glu Asp Gln Glu
                355 360 365
Asp Lys Glu Cys Leu Lys Gln Ala Ile Thr Ala Leu Leu Asn Val Gln
        370 375 380
Ser Gly Met Glu Lys Ile Cys Ser Lys Ser Leu Ala Lys Arg Arg Leu
385 390 395 400
Ser Glu Ser Ala Cys Arg Phe Tyr Ser Gln Gln Met Lys Gly Lys Gln
                                405 410 415
Leu Ala Ile Lys Lys Met Asn Glu Ile Gln Lys Asn Ile Asp Gly Trp
                        420 425 430
Glu Gly Lys Asp Ile Gly Gln Cys Cys Asn Glu Phe Ile Met Glu Gly
                435 440 445
Thr Leu Thr Arg Val Gly Ala Lys His Glu Arg His Ile Phe Leu Phe
        450 455 460
Asp Gly Leu Met Ile Cys Cys Lys Ser Asn His Gly Gln Pro Arg Leu
465 470 475 480
Pro Gly Ala Ser Asn Ala Glu Tyr Arg Leu Lys Glu Lys Phe Phe Met
                                485 490 495
Arg Lys Val Gln Ile Asn Asp Lys Asp Asp Thr Asn Glu Tyr Lys His
                        500 505 510
Ala Phe Glu Ile Ile Leu Lys Asp Glu Asn Ser Val Ile Phe Ser Ala
                515 520 525
Lys Ser Ala Glu Glu Lys Asn Asn Trp Met Ala Ala Leu Ile Ser Leu
        530 535 540
Gln Tyr Arg Ser Thr Leu Glu Arg Met Leu Asp Val Thr Met Leu Gln
545 550 555 560
Glu Glu Lys Glu Glu Gln Met Arg Leu Pro Ser Ala Asp Val Tyr Arg
                                565 570 575
Phe Ala Glu Pro Asp Ser Glu Glu Asn Ile Ile Phe Glu Glu Asn Met
                        580 585 590
Gln Pro Lys Ala Gly Ile Pro Ile Ile Lys Ala Gly Thr Val Ile Lys
                595 600 605
Leu Ile Glu Arg Leu Thr Tyr His Met Tyr Ala Asp Pro Asn Phe Val
        610 615 620
Arg Thr Phe Leu Thr Thr Tyr Arg Ser Phe Cys Lys Pro Gln Glu Leu
625 630 635 640
Leu Ser Leu Ile Ile Glu Arg Phe Glu Ile Pro Glu Pro Glu Pro Thr
                                645 650 655
Glu Ala Asp Arg Ile Ala Ile Glu Asn Gly Asp Gln Pro Leu Ser Ala
                        660 665 670
Glu Leu Lys Arg Phe Arg Lys Glu Tyr Ile Gln Pro Val Gln Leu Arg
                675 680 685
Val Leu Asn Val Cys Arg His Trp Val Glu His His Phe Tyr Asp Phe
        690 695 700
Glu Arg Asp Ala Tyr Leu Leu Gln Arg Met Glu Glu Phe Ile Gly Thr
705 710 715 720
Val Arg Gly Lys Ala Met Lys Lys Trp Val Glu Ser Ile Thr Lys Ile
                                725 730 735
Ile Gln Arg Lys Lys Ile Ala Arg Asp Asn Gly Pro Gly His Asn Ile
                        740 745 750
Thr Phe Gln Ser Ser Pro Pro Thr Val Glu Trp His Ile Ser Arg Pro
                755 760 765
Gly His Ile Glu Thr Phe Asp Leu Leu Thr Leu His Pro Ile Glu Ile
        770 775 780
Ala Arg Gln Leu Thr Leu Leu Glu Ser Asp Leu Tyr Arg Ala Val Gln
785 790 795 800
Pro Ser Glu Leu Val Gly Ser Val Trp Thr Lys Glu Asp Lys Glu Ile
                                805 810 815
Asn Ser Pro Asn Leu Leu Lys Met Ile Arg His Thr Thr Asn Leu Thr
                        820 825 830
Leu Trp Phe Glu Lys Cys Ile Val Glu Thr Glu Asn Leu Glu Glu Arg
                835 840 845
Val Ala Val Val Ser Arg Ile Ile Glu Ile Leu Gln Val Phe Gln Glu
        850 855 860
Leu Asn Asn Phe Asn Gly Val Leu Glu Val Val Ser Ala Met Asn Ser
865 870 875 880
Ser Pro Val Tyr Arg Leu Asp His Thr Phe Glu Gln Ile Pro Ser Arg
                                885 890 895
Gln Lys Lys Ile Leu Glu Glu Ala His Glu Leu Ser Glu Asp His Tyr
                        900 905 910
Lys Lys Tyr Leu Ala Lys Leu Arg Ser Ile Asn Pro Pro Cys Val Pro
                915 920 925
Phe Phe Gly Ile Tyr Leu Thr Asn Ile Leu Lys Thr Glu Glu Gly Asn
        930 935 940
Pro Glu Val Leu Lys Arg His Gly Lys Glu Leu Ile Asn Phe Ser Lys
945 950 955 960
Arg Arg Lys Val Ala Glu Ile Thr Gly Glu Ile Gln Gln Tyr Gln Asn
                                965 970 975
Gln Pro Tyr Cys Leu Arg Val Glu Ser Asp Ile Lys Arg Phe Phe Glu
                        980 985 990
Asn Leu Asn Pro Met Gly Asn Ser Met Glu Lys Glu Phe Thr Asp Tyr
                995 1000 1005
Leu Phe Asn Lys Ser Leu Glu Ile Glu Pro Arg Asn Pro Lys Pro
        1010 1015 1020
Leu Pro Arg Phe Pro Lys Lys Tyr Ser Tyr Pro Leu Lys Ser Pro
        1025 1030 1035
Gly Val Arg Pro Ser Asn Pro Arg Pro Gly Thr Met Arg His Pro
        1040 1045 1050
Thr Pro Leu Gln Gln Glu Pro Arg Lys Ile Ser Tyr Ser Arg Ile
        1055 1060 1065
Pro Glu Ser Glu Thr Glu Ser Thr Ala Ser Ala Pro Asn Ser Pro
        1070 1075 1080
Arg Thr Pro Leu Thr Pro Pro Pro Ala Ser Gly Ala Ser Ser Thr
        1085 1090 1095
Thr Asp Val Cys Ser Val Phe Asp Ser Asp His Ser Ser Pro Phe
        1100 1105 1110
His Ser Ser Asn Asp Thr Val Phe Ile Gln Val Thr Leu Pro His
        1115 1120 1125
Gly Pro Arg Ser Ala Ser Val Ser Ser Ile Ser Leu Thr Lys Gly
        1130 1135 1140
Thr Asp Glu Val Pro Val Pro Pro Pro Val Pro Pro Arg Arg Arg
        1145 1150 1155
Pro Glu Ser Ala Pro Ala Glu Ser Ser Pro Ser Lys Ile Met Ser
        1160 1165 1170
Lys His Leu Asp Ser Pro Pro Ala Ile Pro Pro Arg Gln Pro Thr
        1175 1180 1185
Ser Lys Ala Tyr Ser Pro Arg Tyr Ser Ile Ser Asp Arg Thr Ser
        1190 1195 1200
Ile Ser Asp Pro Pro Glu Ser Pro Pro Leu Leu Pro Pro Arg Glu
        1205 1210 1215
Pro Val Arg Thr Pro Asp Val Phe Ser Ser Ser Pro Leu His Leu
        1220 1225 1230
Gln Pro Pro Pro Leu Gly Lys Lys Ser Asp His Gly Asn Ala Phe
        1235 1240 1245
Phe Pro Asn Ser Pro Ser Pro Phe Thr Pro Pro Pro Pro Gln Thr
        1250 1255 1260
Pro Ser Pro His Gly Thr Arg Arg His Leu Pro Ser Pro Pro Leu
        1265 1270 1275
Thr Gln Glu Val Asp Leu His Ser Ile Ala Gly Pro Pro Val Pro
        1280 1285 1290
Pro Arg Gln Ser Thr Ser Gln His Ile Pro Lys Leu Pro Pro Lys
        1295 1300 1305
Thr Tyr Lys Arg Glu His Thr His Pro Ser Met His Arg Asp Gly
        1310 1315 1320
Pro Pro Leu Leu Glu Asn Ala His Ser Ser
        1325 1330
```

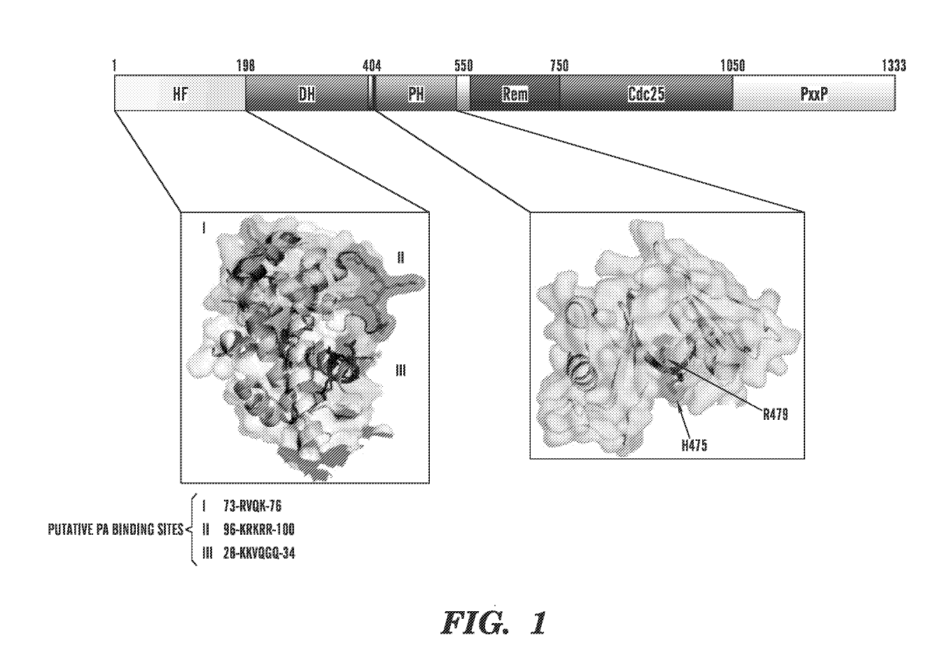

The structure and regulation of Sos is described in Quilliam, “New Insights into the Mechanisms of Sos Activation,” Sci. STKE pe67 (2007), which is hereby incorporated by reference in its entirety. Phosphatidic acid binding sites on Sos are illustrated in FIG. 1. FIG. 1 shows a linker region between the DH and PH domains (residues 404-442) and a linker region between the PH and Rem domains (residues 550-555).

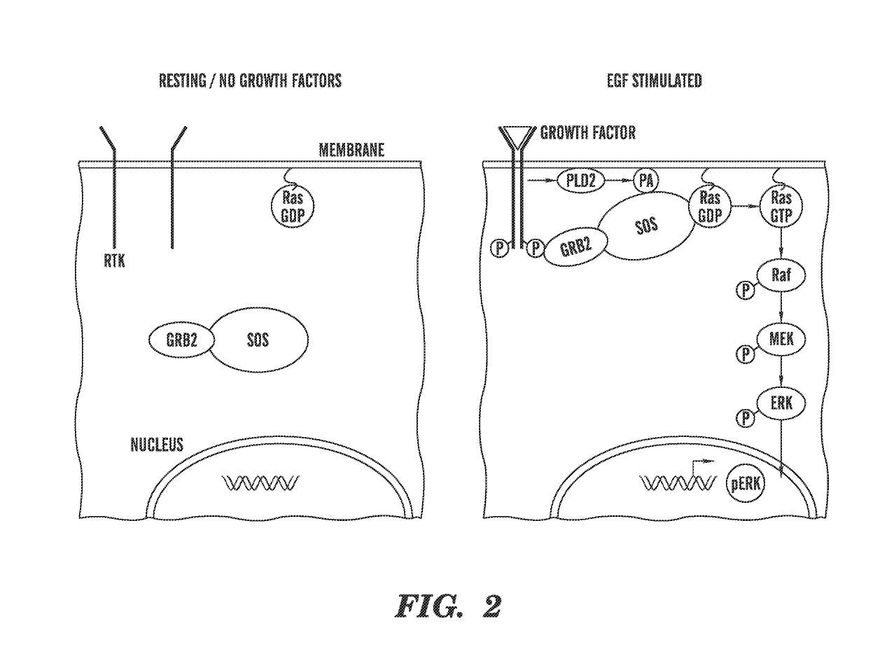

As illustrated in FIG. 2, during resting conditions when there are no growth factors present, receptors are inactivated and there is no MAPK signaling and no Immediate Early gene (“IEG”) (e.g., Fos, Jun, Myc) transcription from the DNA within the nucleus. The activator for the gene transcription MAPK (also called ERK) remains in the cytoplasm in a non-phosphorylated inactive form. The primary reason for this quiescence is the spatial distribution of Sos (the activator of membrane bound Ras) in the cytosol. Ras is active only when bound to GTP, and Sos helps Ras bind to GTP in a signaling-dependent manner. This is reversed upon growth factor stimulation. The receptors are activated (dimerize and transphosphorylate) and Sos is recruited to the membrane where it activates Ras. PLD2 is activated and PA is generated. A cascade of sequential phosphorylation events finally lead to MAPK activation (pERK). pERK travels to the nucleus and brings about transcription of IEGs, which lead to various cellular responses (e.g., growth, proliferation, etc.). The membrane recruitment of Sos is thus highly regulated and coordinated. It is simultaneously recruited to the membrane via GRB2 binding to activated receptors and PLD2 generated PA binding to PH and HF domains. At the membrane, Sos activates Ras, leading to the activation of MAPK signaling cascade.

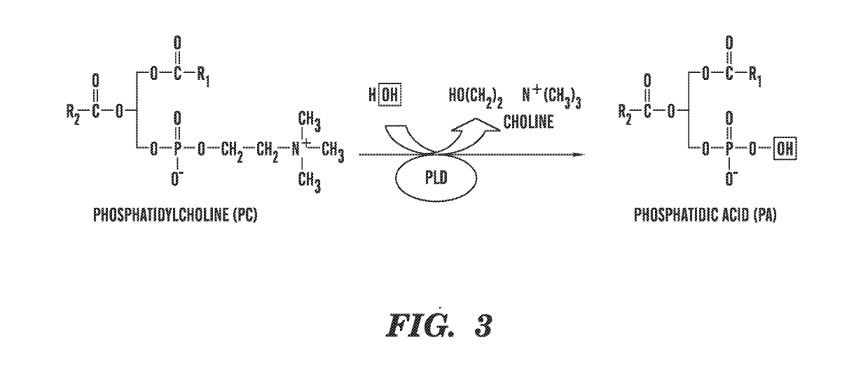

As illustrated in FIG. 3, PLD, which is activated by growth factor stimulation, converts the membrane abundant phosphatidylcholine to phosphatidic acid by removing the choline group. Activation of PLD, and the subsequent generation of phosphatidic acid, is signaling dependent and helps in membrane recruitment and anchorage of Sos.

The gene encoding Son of sevenless has a nucleotide sequence of SEQ ID NO:2, as follows.

| | |
|---|---|
| ccgccgcccc tctccccgcc cagaggcgcc ccgggggcac catgcaggcg cagcagctgc | 60 |
| cctacgagtt tttcagcgaa gagaacgcgc ccaagtggcg gggactactg gtgcctgcgc | 120 |
| tgaaaaaggt ccaggggcaa gttcatccta ctctcgagtc taatgatgat gctcttcagt | 180 |
| atgttgaaga attaattttg caattattaa atatgctatg ccaagctcag ccccgaagtg | 240 |
| cttcagatgt agaggaacgt gttcaaaaaa gtttccctca tccaattgat aaatgggcaa | 300 |
| tagctgatgc ccaatcagct attgaaaaga ggaagcgaag aaacccttta tctctcccag | 360 |
| tagaaaaaat tcatccttta ttaaaggagg tcctaggtta taaaattgac caccaggttt | 420 |
| ctgtttacat agtagcagtc ttagaataca tttctgcaga cattttaaag ctggttggga | 480 |
| attatgtaag aaatatacgg cattatgaaa ttacaaaaca agatattaaa gtggcaatgt | 540 |
| gtgctgacaa ggtattgatg gatatgtttc atcaagatgt agaagatatt aatatattat | 600 |
| ctttaactga cgaagagcct tccacctcag gagaacaaac ttactatgat ttggtaaaag | 660 |
| catttatggc agaaattcga caatatataa gggaactaaa tctaattata aaagttttta | 720 |
| gagagccctt tgtctccaat tcaaaattgt tttcagctaa tgatgtagaa aatatattta | 780 |
| gtcgcatagt agatatacat gaacttagtg taaagttact gggccatata gaagatacag | 840 |
| tagaaatgac agatgaaggc agtccccatc cactagtagg aagctgcttt gaagacttag | 900 |
| cagaggaact ggcatttgat ccatatgaat cgtatgctcg agatattttg cgacctggtt | 960 |
| ttcatgatcg tttccttagt cagttatcaa agcctggggc agcactttat ttgcagtcaa | 1020 |
| taggcgaagg tttcaaagaa gctgttcaat atgttttacc caggctgctt ctggcccctg | 1080 |
| tttaccactg tctccattac tttgaacttt tgaagcagtt agaagaaaaa agtgaagatc | 1140 |
| aagaagacaa ggaatgttta aaacaagcaa taacagcttt gcttaatgtt cagagtggta | 1200 |
| tggaaaaaat atgttctaaa agtcttgcaa aacgaagact gagtgaatct gcatgtcggt | 1260 |
| tttatagtca gcaaatgaag gggaaacaac tagcaatcaa gaagatgaac gagattcaga | 1320 |
| agaatattga tggttgggag ggaaaagaca ttggacagtg ttgtaatgaa tttataatgg | 1380 |
| aaggaactct tacacgtgta ggagccaaac atgagagaca catatttctc tttgatggct | 1440 |
| taatgatttg ctgtaaatca aatcatgggc agccaagact tcctggtgct agcaatgcag | 1500 |
| aatatcgtct taaagaaaag ttttttatgc gaaaggtaca aattaatgat aaagatgaca | 1560 |
| ccaatgaata caagcatgct tttgaaataa ttttaaaaga tgaaaatagt gttatatttt | 1620 |
| ctgccaagtc agctgaagag aaaaacaatt ggatggcagc attgatatct ttacagtacc | 1680 |
| ggagtacact ggaaaggatg cttgatgtaa caatgctaca ggaagagaaa gaggagcaga | 1740 |
| tgaggctgcc tagtgctgat gtttatagat ttgcagagcc tgactctgaa gagaatatta | 1800 |
| tatttgaaga gaacatgcag cccaaggctg gaattccaat tatcaaagca ggaactgtta | 1860 |
| ttaaacttat agagaggctt acgtaccata tgtacgcaga tcccaatttt gttcggacat | 1920 |
| ttcttacaac atacagatcc ttttgcaaac ctcaagaact actgagtctt ataatagaaa | 1980 |
| ggtttgaaat tccagagcct gagccaacag aagctgatcg catagctata gagaatggag | 2040 |
| atcaaccctt gagtgcagaa ctgaaaagat ttagaaaaga atatatacag cctgtgcaac | 2100 |
| tgcgagtatt aaatgtatgt cggcactggg tagagcacca cttctatgat tttgaaagag | 2160 |
| atgcatatct tttgcaacga atggaagaat ttattggaac agtaagaggt aaagcaatga | 2220 |
| aaaaatgggt tgaatccatc actaaaataa tccaaaggaa aaaaattgca agagacaatg | 2280 |
| gaccaggtca taatattaca tttcagagtt cacctcccac agttgagtgg catataagca | 2340 |
| gacctgggca catagagact tttgacctgc tcaccttaca cccaatagaa attgctcgac | 2400 |
| aactcacttt acttgaatca gatctatacc gagctgtaca gccatcagaa ttagttggaa | 2460 |
| gtgtgtggac aaaagaagac aaagaaatta actctcctaa tcttctgaaa atgattcgac | 2520 |
| ataccaccaa cctcactctg tggtttgaga aatgtattgt agaaactgaa aatttagaag | 2580 |
| aaagagtagc tgtggtgagt cgaattattg agattctaca agtctttcaa gagttgaaca | 2640 |
| actttaatgg tgtccttgag gttgtcagtg ctatgaattc atcacctgtt tacagactag | 2700 |
| accacacatt tgagcaaata ccaagtcgcc agaagaaaat tttagaagaa gctcatgaat | 2760 |
| tgagtgaaga tcactataag aaatatttgg caaaactcag gtctattaat ccaccatgtg | 2820 |
| tgcctttctt tggaatttat ctcactaata tcttgaaaac agaagaaggc aaccctgagg | 2880 |
| tcctaaaaag acatggaaaa gagcttataa actttagcaa aaggaggaaa gtagcagaaa | 2940 |
| taacaggaga gatccagcag taccaaaatc agccttactg tttacgagta gaatcagata | 3000 |
| tcaaaaggtt ctttgaaaac ttgaatccga tgggaaatag catggagaag gaatttacag | 3060 |
| attatctttt caacaaatcc ctagaaatag aaccacgaaa ccctaagcct ctcccaagat | 3120 |
| ttccaaaaaa atatagctat cccctaaaat ctcctggtgt tcgtccatca aacccaagac | 3180 |
| caggtaccat gaggcatccc acacctctgc agcaggagcc aaggaaaatt agttatagta | 3240 |
| ggatccctga aagtgaaaca gaaagtacag catctgcacc aaattctcca agaacaccgt | 3300 |
| taacacctcc gcctgcttct ggtgcttcca gtaccacaga tgtttgcagt gtatttgatt | 3360 |
| ccgatcattc gagccctttt cactcaagca atgataccgt ctttatccaa gttactctgc | 3420 |
| cccatggccc aagatctgct tctgtatcat ctataagttt aaccaaaggc actgatgaag | 3480 |
| tgcctgtccc tcctcctgtt cctccacgaa gacgaccaga atctgcccca gcagaatctt | 3540 |
| caccatctaa gattatgtct aagcatttgg acagtccccc agccattcct cctaggcaac | 3600 |
| ccacatcaaa agcctattca ccacgatatt caatatcaga ccggacctct atctcagacc | 3660 |
| ctcctgaaag ccctccctta ttaccaccac gagaacctgt gaggacacct gatgttttct | 3720 |
| caagctcacc actacatctc caacctcccc ctttgggcaa aaaaagtgac catggcaatg | 3780 |
| ccttcttccc aaacagccct tcccccttta caccacctcc tcctcaaaca ccttctcctc | 3840 |
| acggcacaag aaggcatctg ccatcaccac cattgacaca agaagtggac cttcattcca | 3900 |
| ttgctgggcc gcctgttcct ccacgacaaa gcacttctca acatatccct aaactccctc | 3960 |
| caaaaactta caaaagggag cacacacacc catccatgca cagagatgga ccaccactgt | 4020 |
| tggagaatgc ccattcttcc tgagttcctc tgtactggga tgtatatttt cctagcccca | 4080 |
| aatccattgc tggcaatgga tgcactgaat gtgccagcac tgaggagtta aaatgagaac | 4140 |
| tccaaacact aacgactctt cttcaagatg cagtataaga caatgaattt taacctagat | 4200 |
| gtaattatac aatggaaatg gtattccagt ttagaatatg gaaagaccga cctagaggaa | 4260 |
| ttggacaact gattgcacct gaaaatcata aagggacttt ttctggccaa taggcaggag | 4320 |
| tcctcttttt gtgaagtgat cttttgtgaa gtgatcatta aagggatgga aaacacagtc | 4380 |
| tagtgtccag caggcccaca tgacagtttt tgtaattcaa attatgcact tttaaaaaaa | 4440 |
| aaaacttaaa cagggatctt aatatcttcc tttgttttcc tttgctttac tcttctactt | 4500 |
| tagaatattt tcttaaaaat cactcaaagg actgtgagga aaggctgtgg tacctgacct | 4560 |
| tgttgaaatc aaggcccggc actgtactac aggcctgttt acagattatt acggtgaact | 4620 |
| gaatgggtac cgaggcttca ccaaagaggt acttttttgt tgttgttgtt gttttaggaa | 4680 |
| taattgtacc aattttaaga gcattccccc cacctgtccc cacacaccca aacaaaatgt | 4740 |
| ggtggtgttg ccttcaaaaa agagaagttt tgtgtcatta acatgacaga agaacttttt | 4800 |
| aaaaaaaaat aactgtcaac tattctattt gcatttagga gactgttcat ctatgctaga | 4860 |
| ttgtcatttt ccctccttct cccacagaag tttactggta gtccatgtca tggctcgtag | 4920 |
| ctatccctct aaccatacca tggaaatgca ggcacccaat gtgaaaagga gcacttgctg | 4980 |
| ggcatcactg acaccgctca tgttttacac atagttgagt aatcagcata tctagaatta | 5040 |
| tcttgcattg cctaaatcat atgtatatag tgaatgttat ataatatacc tggcaggtct | 5100 |
| gttttaattt aattgaataa agatacaaat actttgtttg gctggcatat attaaattat | 5160 |
| tatatgaaga aaatggttga ttcttatttc ttttagttga aaaacacaca aaaacactaa | 5220 |
| acataaaagc acgttttgtg gtacgccttg gtttctagtt ctggatatat gtattaatct | 5280 |
| taaagaatga tctaaagtca cagcttggca tataggaaga tacttggtta gaataggatg | 5340 |
| ggtatctttt aagcagaact ttgtaagtaa gttttaagtt cctaagcaac aagtatacag | 5400 |
| tttgttaagg tttatattga acattctacc ttgcttgagg ttttctgctt tatcaaaact | 5460 |
| attcataata gaacatctac ctgcatcatt actatcataa tatggattat tcataaagcg | 5520 |
| tatgtactgt tcttctgagt ctaacaaata ctgtgactga aatgcatgca ccttgttgaa | 5580 |
| gagaaaacat tttattaatc tccactataa tgtctgtgaa atgtgcagta ttccatcctt | 5640 |
| agagagttta tctggttcat cagcatctgc atgtatcctc tgaataatga agcaacattt | 5700 |
| cagcaattct gttaaccaca ccattagaga catttttaga agtgctgcaa catggtggga | 5760 |
| acctagtact ttaataattt cctatgatat ttattagagc ataaaatgta catcacgata | 5820 |
| gtacagtatg tagtccttta agaccagaag gtatgttaac tgcaccatca ttaagcagtc | 5880 |
| tatcctattt tcttatttat ttgttagaat tgaatccttt ctcaatatct gtaccattcc | 5940 |
| tatgatggta gccattcttc tctttataaa atatttcctc tttgaaaagc gtcctaaaac | 6000 |
| attaatttca aatcccctct tggatgcaaa gtgtccctaa gaccatccat tcaacagtat | 6060 |
| ttatgtgagc caacttacta gagccgctac aactaatcat gatagacagt ccagaatctc | 6120 |
| cctggacttt atactgggcc agcctcattg tccctaagat ttctcttttt atgtctcatg | 6180 |
| gattacttct ctttttaatt gccccaggca cattccaaat gagaacctgg taaaagtcac | 6240 |
| attggacccc cttcagtatg tcttcggcta atgtgttagc cttcaataag gtgataaccc | 6300 |
| cttttcaacc tatctagtct ataaagagga aaaattcaga tttggagttc taatcctatt | 6360 |
| tctcccaaag ggaactattc ttctacccca tggttctagg agaccacttc actgtgcaac | 6420 |
| agttttacat gcattgtgga tggaggatat ttttattctg aactgttaca tttaggtaaa | 6480 |
| aatattttct gcagtggtaa ctgattagaa aaatgccaat tggatgccct taggtggagg | 6540 |
| tgagaaaatg gcatccttgc cttcttctca atatgaaaca ttaactagtt gacaaattta | 6600 |
| tccttgtagt aatgaaaatc tatttaatca gggaccagaa atggctgagg agataaatgc | 6660 |
| atcattacaa aattctgctt ttgaatcctg gacattacaa gggggtaaat gcagcatgac | 6720 |
| tttttgttaa ccacattcca aaatgtggaa catttctttt agaaatgaaa atatttcaag | 6780 |
| gctgatgtat tttaagacta cacattatca ggaaacatac attgagagtt cgcttaatta | 6840 |
| aaggttgttg ggcatcaaat tatgtttagt aggttactat tctctaacaa ctcaaggatg | 6900 |
| ctttaatgga tctgaatttg acaaagagca tgccacacta atactacagt caacaacagc | 6960 |
| ccagagaaca attactatgt cagctggagg ctatattatg attctaaatt cttaaaggtt | 7020 |
| tttttccctc cataaatcaa aaattacctt atgtaaacca aaaattagtt ggtatttatg | 7080 |
| gtcatgatct taattctcaa gtttagctta atcttgtatt tcattgtttg tcttctaata | 7140 |
| tgacagctta aattcagatt tttaagtgac tcagcaaaat aggaggagtg tcccaattta | 7200 |
| ttagtgttgt acatattgaa gaaaaccttt ttgttccttc agatttagaa agaaacagtt | 7260 |
| taaccattta tttcttggta ttctgctgct gtgaataggt ttacttattc ttttaacata | 7320 |
| tattgtgtga ctacgcaagt ataggtcctg ttgtattgca ttaatcttta ccagtaacta | 7380 |
| aacatcacaa ggttaatatt ggtttggctg aaagaattat gcagtaaagt tatttataag | 7440 |
| ggaacatgat gactttattc aatatttttc ttctttgaaa catctcatta ctaactttta | 7500 |
| agattatttc ataatccctt atacatgagc caatgaaata ttttgagctc tacttaagaa | 7560 |
| gcatgaagtc tatattataa atctaaacaa caaaagcact tgtaacttgt ttagtaaatt | 7620 |
| ccatgcctta ttttccattt ttgacaccgt aaagtgcatt ttctgtcgac tgtgagcaaa | 7680 |
| tttccctttt gcctttaatg aaaatagtgc attaagtagc caattgctgc agaattataa | 7740 |
| agcaaattag ctaacagtgg gtcacattgg actaccactc aaagaaacaa gatgcataac | 7800 |
| agatgatgga aggtacatgt ggtgtggctt agggagagcg cacctgtgcc agatgcatgc | 7860 |
| cactttcagt agctgccaaa tgtgccttta attaagaaca tctctaattt ttttcttcag | 7920 |
| atcaagtcag catttggggt cgggatttgg gcagggaact ttggggtgca aatatctggt | 7980 |
| gaaaccacca gagaatttgt ttgtcaaagt gacatgtata attttagttg agacgtgttt | 8040 |
| gtgtatatat gtgtatttgc ctttgtttag tagcacggtt atttgctaaa ataattgtca | 8100 |
| aaaaattgct ctgtcggtcg gtctttgatg taagaacaaa gtgtggtcta tacatctaat | 8160 |
| atggcctttg tacctaacca tgttcatagg taatctttgt actctgtgtg cagcagtatt | 8220 |
| tggtttgcat ttaaagtgaa aataacggct gttatttttc tggcattact aagataaact | 8280 |
| gaaaaataat aaagaaaaat gccttacata gcccacaaaa aaaaaaaaaa a | 8331 |

One aspect of the present invention relates to a method of controlling pleckstrin homology domain-dependent membrane recruitment of Son of sevenless or histone folds domain-dependent membrane recruitment of Son of sevenless. This method involves selecting a cell where control of pleckstrin homology domain membrane recruitment of Son of sevenless or histone folds domain-dependent membrane recruitment of Son of sevenless is needed. Binding of Son of sevenless to phosphatidic acid is modulated in the cell under conditions effective to control pleckstrin homology domain-dependent membrane recruitment of Son of sevenless or histone folds domain-dependent membrane recruitment of Son of sevenless.

Selecting a cell where control of pleckstrin homology domain membrane recruitment of Son of sevenless or histone folds domain-dependent membrane recruitment of Son of sevenless is needed can be based on evidence for changes in Sos-dependent Ras activity as measured by GTP binding status, as further described in the Examples.

Binding of Son of sevenless to phosphatidic acid according to the methods of the present invention may be enzyme phospholipase D2-mediated. Accordingly, modulating binding of Son of sevenless to phosphatidic acid may be carried out with an inhibitor of binding of Son of sevenless to phosphatidic acid, where binding of Son of sevenless to phosphatidic acid is enzyme phospholipase D2-mediated.

Suitable inhibitors may either bind to enzyme phospholipase D2 or Son of sevenless. When the inhibitor binds to Son of sevenless, binding preferably occurs at histidine 475 and/or arginine 479 of Son of sevenless. Binding may also preferably occur at the putative PA binding sites on the histone folds.

In an alternative embodiment, modulating binding of Son of sevenless to phosphatidic acid is carried out with an activator of binding of Son of sevenless to phosphatidic acid. According to this embodiment, binding of Son of sevenless to phosphatidic acid may be phospholipase D2-mediated.

Modulating binding of Son of sevenless to phosphatidic acid may be carried out with a variety of agents including, without limitation, an antibody, an antibody binding fragment, a small molecule, or a nucleic acid.

When employed, antibodies may be either monoclonal antibodies or polyclonal antibodies. Monoclonal antibody production may be carried out by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler et al., Nature 256:495 (1975), which is hereby incorporated by reference in its entirety.

Mammalian lymphocytes are immunized by in vivo immunization of the animal (e.g., a mouse) with the desired protein or polypeptide. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (Milstein et al., Eur. J. Immunol. 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering a target protein or polypeptide subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in Harlow et. al., editors, Antibodies: A Laboratory Manual (1988), which is hereby incorporated by reference in its entirety.

It is also possible to use the anti-idiotype technology to produce monoclonal antibodies that mimic an epitope. As used in this invention, “epitope” means any antigenic determinant on an antigen to which the paratope of an antibody binds. Epitopic determinants usually consist of chemically active surface groupings of molecules, such as amino acids or sugar side chains and usually have specific three dimensional structural characteristics, as well as specific charge characteristics. For example, an anti-idiotype monoclonal antibody made to a first monoclonal antibody will have a binding domain in the hypervariable region that is the image of the epitope bound by the first monoclonal antibody.

In addition to utilizing whole antibodies, the present invention may employ the use of binding portions of such antibodies. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, pp. 98-118 (N.Y. Academic Press, 1983), which is hereby incorporated by reference in its entirety.

Suitable agents for modulating binding of Son of sevenless to phosphatidic acid may also include aptamers. Aptamers are single-stranded, partially single-stranded, partially double-stranded, or double-stranded nucleotide sequences, advantageously a replicatable nucleotide sequence, capable of specifically recognizing a selected nonoligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides and nucleotides comprising backbone modifications, branchpoints and normucleotide residues, groups or bridges. Aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof, and/or corresponding complementary sequence, promoter, or primer-annealing sequence needed to amplify, transcribe, or replicate all or part of the aptamer molecule or sequence.

Nucleic acid aptamers include multivalent aptamers and bivalent aptamers. Methods of making bivalent and multivalent aptamers and their expression in multi-cellular organisms are described in U.S. Pat. No. 6,458,559 to Shi et al., which is hereby incorporated by reference in its entirety. A method for modular design and construction of multivalent nucleic acid aptamers, their expression, and methods of use are described in U.S. Patent Publication No. 2005/0282190, which is hereby incorporated by reference in its entirety. Aptamers may be designed to modulate binding of Son of sevenless to phosphatidic acid.

Identifying suitable nucleic acid aptamers involves selecting aptamers that bind enzyme phospholipase D2 or Son of sevenless with sufficiently high affinity (e.g., K_{d}=20-50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell. Biol. 17:1649-1657 (1997), which is hereby incorporated by reference in its entirety).

Using these same procedures, suitable nucleic acid aptamers of the present invention that inhibit binding of Son of sevenless to phosphatidic acid can also be identified.

For example, identifying suitable nucleic acid aptamers can be carried out using an established in vitro selection and amplification scheme known as SELEX. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk & Gold, “Systematic Evolution of Ligands by Exponential Enrichment: RNA Ligands to Bacteriophage T4 DNA Polymerase,” Science 249:505-510 (1990), which are hereby incorporated by reference in their entirety. The SELEX procedure can be modified so that an entire pool of aptamers with binding affinity can be identified by selectively partitioning the pool of aptamers. This procedure is described in U.S. Patent Application Publication No. 2004/0053310, which is hereby incorporated by reference in its entirety.

Aptamers may be identified using screening assays such as yeast-two hybrid approaches described in U.S. Patent Application Serial No. 20040210040 to Landolfi et al., which is hereby incorporated by reference in its entirety. Other approaches, including those described herein, can also be used.

Suitable nucleic acid agents also include siRNA, shRNA, microRNA, antisense RNA, and engineered genes encoding a therapeutic nucleic acid or polypeptide.

Antisense molecules and their use for inhibiting gene expression are well known in the art (see, e.g., Cohen, 1989, In: Oligodeoxyribonucleotides, Antisense Inhibitors of Gene Expression, CRC Press), which is hereby incorporated by reference in its entirety. Antisense nucleic acids are DNA or RNA molecules that are complementary to at least a portion of a specific mRNA molecule (Weintraub, 1990, Scientific American 262:40). In the cell, antisense nucleic acids hybridize to the corresponding mRNA, in this case mRNA for either enzyme phospholipase D2 or Son of sevenless, forming a double-stranded molecule thereby inhibiting the translation of genes.

The use of antisense methods to inhibit the translation of genes is known in the art, and is described, for example, in Marcus-Sakura, Anal. Biochem. 172:289 (1988), which is hereby incorporated by reference in its entirety. Such antisense molecules may be provided to the cell via genetic expression using DNA encoding the antisense molecule as taught by U.S. Pat. No. 5,190,931 to Inoue, which is hereby incorporated by reference in its entirety.

Alternatively, antisense molecules of the invention may be made synthetically. Antisense oligomers of about 15 nucleotides are preferred, since they are easily synthesized and introduced into a target cell. Synthetic antisense molecules contemplated by the invention include oligonucleotide derivatives known in the art which have improved biological activity compared to unmodified oligonucleotides (U.S. Pat. No. 5,023,243 to Tullis, which is hereby incorporated by reference in its entirety).

Another example of such an agent is an siRNA targeted to the phospholipase D2 or Son of sevenless nucleotide sequence, which interferes with translation of these proteins. Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), which is hereby incorporated by reference in its entirety). In RNAi, the introduction of double stranded RNA (dsRNA, or iRNA, for interfering RNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22-, or 23-nucleotide RNAs (siRNA) by a putative RNAaseIII-like enzyme (Tuschl, “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3, (2000), which are hereby incorporated by reference in their entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21- and 22-Nucleotide RNAs,” Gene Dev. 15(2):188-200 (2001), which is hereby incorporated by reference in its entirety). The dsRNA for enzyme phospholipase D2 or Son of sevenless can be generated by transcription in vivo, which involves modifying the nucleic acid molecule encoding enzyme phospholipase D2 or Son of sevenless for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. Alternatively, complementary sense and antisense RNAs derived from a substantial portion of the coding region of the enzyme phospholipase D2 or Son of sevenless nucleic acid molecule are synthesized in vitro (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc Natl Acad Sci USA 95:15502-15507; Tabara et al., “RNAi in C. elegans: Soaking in the Genome Sequence,” Science 282:430-431 (1998), which are hereby incorporated by reference in its entirety). The resulting sense and antisense RNAs are annealed in an injection buffer, and dsRNA is administered to the subject using any method of administration described herein.

siRNA and shRNA can be administered to a subject systemically as described herein or otherwise known in the art. Systemic administration can include those methods described above, but preferably intravenous, intraarterial, subcutaneous, intramuscular, catheterization, or nasopharyngeal as is generally known in the art. Alternatively, the siRNA or shRNA can be administered to a subject locally or to local tissues as described herein or otherwise known in the art. Local administration can include, for example, catheterization, implantation, direct injection, stenting, or portal vein administration to relevant tissues, or any other local administration technique, method or procedure, as is generally known in the art.

siRNA or shRNA is preferably administered alone or as a component of a composition. Suitable compositions include the siRNA or shRNA formulated or complexed with polyethylenimine (e.g., linear or branched PEI) and/or polyethylenimine derivatives, including for example, grafted PEIs such as galactose PEI, cholesterol PEI, antibody derivatized PEI, and polyethylene glycol PEI (PEG-PEI) derivatives thereof (see, e.g., Ogris et al., AAPA Pharm Sci 3:1-11 (2001); Furgeson et al., Bioconjugate Chem. 14:840-847 (2003); Kunath et al., Pharmaceutical Res 19:810-817 (2002); Choi et al., Bull. Korean Chem. Soc. 22:46-52 (2001); Bettinger et al., Bioconjugate Chem. 10:558-561 (1999); Peterson et al., Bioconjugate Chem. 13:845-854 (2002); Erbacher et al., J. Gene Medicine Preprint 1:1-18 (1999); Godbey et al., Proc Natl Acad Sci USA 96:5177-5181 (1999); Godbey et al., J Controlled Release 60:149-160 (1999); Diebold et al., J Biol Chem 274:19087-19094 (1999); Thomas & Klibanov, Proc Natl Acad Sci USA 99:14640-14645 (2002); and U.S. Pat. No. 6,586,524 to Sagara, which are hereby incorporated by reference in their entirety.

The siRNA or shRNA molecule can also be present in the form of a bioconjugate, for example a nucleic acid conjugate as described in U.S. Pat. No. 6,528,631, U.S. Pat. No. 6,335,434, U.S. Pat. No. 6,235,886, U.S. Pat. No. 6,153,737, U.S. Pat. No. 5,214,136, or U.S. Pat. No. 5,138,045, which are hereby incorporated by reference in their entirety.

In addition to using an antisense molecule to inhibit expression of a nucleic acid encoding enzyme phospholipase D2 or Son of sevenless, the present invention encompasses the use of ribozymes in this manner. Ribozymes are another nucleic acid that may be transfected into a cell to inhibit nucleic acid expression in the cell. Ribozymes and their use for inhibiting gene expression are also well known in the art (see, e.g., Cech et al., J. Biol. Chem. 267:17479-17482 (1992); Hampel et al., Biochemistry 28:4929-4933 (1989); WO 92/07065; U.S. Pat. No. 5,168,053 to Altman, which are hereby incorporated by reference in their entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, J. Amer. Med. Assn. 260:3030 (1988), which is hereby incorporated by reference in its entirety). A major advantage of this approach is that, because they are sequence-specific, only mRNAs with particular sequences are inactivated.

There are two basic types of ribozymes, namely, tetrahymena-type (Hasselhoff, Nature 334:585 (1988), which are hereby incorporated by reference in its entirety) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of the proteins of interest may be designed by incorporating target sequences into the basic ribozyme structure which are complementary to the mRNA sequence of the nucleic acid encoding the protein of interest. Ribozymes targeting enzyme phospholipase D2 or Son of sevenless may be synthesized using commercially available reagents (Applied Biosystems, Inc., Foster City, Calif.) or they may be expressed from DNA encoding them.

In addition to the foregoing, it is also contemplated that the nucleic acids encoding peptides or aptamers or antisense materials can be delivered according to gene therapy approaches for expression in vivo of the peptides or aptamers or antisense nucleic acids, whereby such expression thereof can inhibit activity of enzyme phospholipase D2 or Son of sevenless. Thus, naked DNA or infective transformation vectors can be used for delivery, whereby the naked DNA or infective transformation vector contains a recombinant gene that encodes the peptide or RNA. The peptide or RNA molecule is then expressed in the transformed cell, and inhibits activity of enzyme phospholipase D2 or Son of sevenless.

The recombinant gene includes, operatively coupled to one another, an upstream promoter operable in mammalian cells and optionally other suitable regulatory elements (i.e., enhancer or inducer elements), a coding sequence that encodes the therapeutic aptamer or peptide, and a downstream transcription termination region. Any suitable constitutive promoter or inducible promoter can be used to regulate transcription of the recombinant gene, and one of skill in the art can readily select and utilize such promoters, whether now known or hereafter developed. The promoter can also be specific for expression in certain tissues where enzyme phospholipase D2 or Son of sevenless are to be affected. Tissue specific promoters can also be made inducible/repressible using, e.g., a TetO response element. Other inducible elements can also be used. Known recombinant techniques can be utilized to prepare the recombinant gene, transfer it into the expression vector (if used), and administer the vector or naked DNA to a patient. Exemplary procedures are described in Sambrook and Russell, Molecular Cloning: A Laboratory Manual (2d ed. 1989), which is hereby incorporated by reference in its entirety. One of skill in the art can readily modify these procedures, as desired, using known variations of the procedures described therein.

Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988); Rosenfeld et al., Science 252:431-434 (1991); PCT Publication No. WO 93/07283; PCT Publication No. WO 93/06223; and PCT Publication No. WO 93/07282, which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), which are hereby incorporated by reference in their entirety.

Retroviral vectors that have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, which are hereby incorporated by reference in their entirety.

As a further alternative, “synthetic antibodies” can be generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The invention thus includes an isolated DNA encoding an anti-enzyme phospholipase D2 or anti-Son of sevenless antibody, or DNA encoding a portion of the antibody.

To isolate DNA encoding an antibody, for example, DNA is extracted from antibody expressing phage obtained as described herein. Such extraction techniques are well known in the art and are described, for example, in Sambrook et al., Molecular Cloning: A Laboratory Manual, Cold Springs Laboratory, Cold Springs Harbor, N.Y. (1989); Ausubel et al., “Short Protocols in Molecular Biology,” New York:Wiley (1999), which are hereby incorporated by reference in their entirety.

Another form of antibody includes a nucleic acid sequence which encodes the antibody and which is operably linked to promoter/regulatory sequences which can direct expression of the antibody in vivo. For a discussion of this technology, see, for example, Cohen, Science 259:1691-1692 (1993); Fynan et al. Proc. Natl. Acad. Sci. 90:11478-11482 (1993); and Wolff et al. Biotechniques 11:474-485 (1991), which are hereby incorporated by reference in their entirety), which describe the use of naked DNA as an antibody/vaccine. For example, a plasmid containing suitable promoter/regulatory sequences operably linked to a DNA sequence encoding an antibody may be directly administered to a patient using the technology described in the aforementioned references.

Alternatively, the promoter/enhancer sequence operably linked to DNA encoding the antibody may be contained within a vector, which vector is administered to a patient. The vector may be a viral vector which is suitable as a delivery vehicle for delivery of the DNA encoding the antibody to the patient, or the vector may be a non-viral vector which is suitable for the same purpose. Examples of viral and non-viral vectors for delivery of DNA to cells and tissues are described above.

Another aspect of the present invention relates to a method of controlling Ras. This method involves selecting a cell where control of Ras is needed and modulating binding of Son of sevenless to phosphatidic acid in the cell under conditions effective to control Ras.

Selecting a cell where control of Ras is needed can be carried out on the basis of altered biological behavior as a result of Sos-mediated hypo- or hyper-activation of Ras as measured by guanine nucleotide binding status.

A further aspect of the present invention relates to a method of treating a subject for a condition mediated by Ras. This method involves selecting a subject having a condition mediated by Ras and modulating binding of Son of sevenless to phosphatidic acid in the subject under conditions effective to treat the condition mediated by Ras.

Conditions mediated by Ras involve cell proliferation, differentiation, motility, death, and/or cell survival. In particular, conditions mediated by Ras involve cancer. Cancer includes, without limitation, bladder cancer, renal cancer, breast cancer, colon cancer, prostate cancer, lung cancer, skin cancer, pancreas cancer, and liver cancer. Conditions mediated by Ras also encompasses premalignant conditions to stop the progression of, or cause regression of, the premalignant conditions. Examples of premalignant conditions include hyperplasia, dysplasia, and metaplasia. Other conditions mediated by Ras in accordance with this aspect of the present application include Noonan syndrome, Hereditary Gingival Fibromatosis Type I, and multi-drug resistance.

In practicing the methods of treating a subject for a condition mediated by Ras of the present invention, the method involves modulating binding of Son of sevenless to phosphatidic acid in the subject under conditions effective to treat the condition mediated by Ras. In one embodiment, modulating binding of Son of sevenless to phosphatidic acid in the subject involves administering to the subject an agent that inhibits or activates binding of Son of sevenless to phosphatidic acid. Administering may be carried out by administering an agent orally, intradermally, intramuscularly, intraperitoneally, intravenously, subcutaneously, or intranasally. The agent of the present invention may be administered alone or with suitable pharmaceutical carriers, and can be in solid or liquid form, such as tablets, capsules, powders, solutions, suspensions, or emulsions.

The agent may be orally administered, for example, with an inert diluent, or with an assimilable edible carrier, or it may be enclosed in hard or soft shell capsules, or it may be compressed into tablets, or it may be incorporated directly with food. For oral therapeutic administration, the agent of the present invention may be incorporated with excipients and used in the form of tablets, capsules, elixirs, suspensions, syrups, and the like. Such compositions and preparations should contain at least 0.1% of the agent. The percentage of the agent in these compositions may, of course, be varied and may conveniently be between about 2% to about 60% of the weight of the unit. The amount of agent in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agent of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

Agents may also be administered directly to the airways in the form of an aerosol. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Suitable subjects for this aspect of the present invention include, without limitation, any mammal, preferably a human.

Yet another aspect of the present invention relates to a method of identifying compounds potentially effective in treating a condition mediated by Ras. This method involves providing one or more candidate compounds and contacting each of the candidate compounds with a cell. The effect of the candidate compounds on binding Son of sevenless to phosphatidic acid is evaluated. Candidate compounds which modulate binding of Son of sevenless to phosphatidic acid are identified as compounds potentially effective in treating a condition mediated by Ras.

In carrying out this method, a cell is provided which expresses Son of sevenless and/or enzyme phospholipase D2. To this end, a nucleic acid molecule encoding a Son of sevenless and/or enzyme phospholipase D2 polypeptide or protein can be introduced into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted Son of sevenless and/or enzyme phospholipase D2 protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the Son of sevenless and/or enzyme phospholipase D2-encoding sequence in a cell. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes.

Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The Son of sevenless and/or enzyme phospholipase D2-encoding nucleic acid, a promoter molecule of choice, a suitable 3′ regulatory region, and if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a Son of sevenless and/or enzyme phospholipase D2 protein is inserted into a vector in the sense (i.e., 5′→3′) direction, such that the open reading frame is properly oriented for the expression of the encoded Son of sevenless and/or enzyme phospholipase D2 protein under the control of a promoter of choice. Single or multiple nucleic acids may be ligated into an appropriate vector in this way, under the control of a suitable promoter, to prepare a nucleic acid construct.

Once the isolated nucleic acid molecule encoding the Son of sevenless and/or enzyme phospholipase D2 protein or polypeptide has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

In all aspects of the present invention “contacting each of the candidate compounds with a cell” can be carried out as desired, including, but not limited to, in culture in a suitable growth medium for the cell. Alternatively, mice, rats or other mammals are injected with compounds to be selected.

Methods of identifying compounds potentially effective in treating a condition mediated by Ras can also be carried out in a cell-free format.

In one embodiment, the assay is directed to the identification of a compound that modulate binding of Son of sevenless to phosphatidic acid. This method involves combining Son of sevenless (i.e., a biologically active portion thereof), PLD2 (i.e., a biologically active portion thereof), and/or phosphatidic acid in the presence of a test compound, under conditions effective to allow binding of Son of sevenless to phosphatidic acid; and then measuring the binding of Son of sevenless to phosphatidic acid. Upon comparing the measured binding of binding of Son of sevenless to phosphatidic acid in the absence of the test compound with the measured binding of Son of sevenless to phosphatidic acid in the presence of the test compound, it is possible to assess the efficacy of the test compound. Where a decrease in binding occurs, the test compound inhibits binding of Son of sevenless to phosphatidic acid.

Detection of binding can be achieved through any suitable procedure that is known in the art or hereafter developed. Exemplary procedures for use in a cell-free format include, without limitation, a competitive binding assay, direct measurement, or detecting changes in e.g., the activity of pleckstrin homology domain-dependent membrane recruitment of Son of sevenless, histone folds domain-dependent membrane recruitment of Son of sevenless, and/or control of Ras (all indirect measures of binding of Son of sevenless to phosphatidic acid). As for the binding that is to be detected, either binding of the test compound to Son of sevenless or binding of the test compound to PLD2 enzyme can be measured.

Binding of a test compound to Son of sevenless, PLD2, or interaction of Son of sevenless with phosphatidic acid in the presence and absence of a candidate test compound, can be accomplished in any vessel suitable for containing the reactants. Examples of such vessels include, without limitation, microtiter plates, test tubes, and micro-centrifuge tubes.

In one approach, a fusion protein can be provided which adds a domain that allows one or both of Son of sevenless and PLD2 to be bound to a matrix. For example, glutathione-S-transferase/Son of sevenless fusion proteins or glutathione-S-transferase/PLD2 fusion proteins can be adsorbed onto glutathione sepharose beads (Sigma Chemical; St. Louis, Mo.) or glutathione derivatized microtiter plates, which are then combined with the test compound or the test compound and either the non-adsorbed Son of sevenless or PLD2, and the mixture incubated under conditions conducive to complex formation (e.g., at physiological conditions for salt and pH). Following incubation, the beads or microtiter plate wells are washed to remove any unbound components, the matrix immobilized in the case of beads, and complex determined either directly or indirectly, for example, as described above. Alternatively, the complexes can be dissociated from the matrix, and the level of Son of sevenless binding or activity determined using standard techniques.

Other techniques for immobilizing proteins on matrices can also be used in the screening assays of the invention. For example, either Son of sevenless or PLD2 can be immobilized utilizing conjugation of biotin and streptavidin. Biotinylated Son of sevenless or PLD2 can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with Son of sevenless or PLD2, but which do not interfere with their binding, can be derivatized to the wells of the plate, and unbound Son of sevenless or PLD2 trapped in the wells by antibody conjugation. Methods for detecting such complexes, in addition to those described above for the GST-immobilized complexes, include immunodetection of complexes using antibodies reactive with the Son of sevenless or PLD2, as well as enzyme-linked assays which rely on detecting an enzymatic activity associated with the Son of sevenless or PLD2.

### EXAMPLES

The Examples set forth below are for illustrative purposes only and are not intended to limit, in any way, the scope of the present invention.

### Example 1

### Phospholipase D2-generated PA Couples EGFR Stimulation to Ras Activation by Sos

General Reagents

1,2-Dilauroyl-sn-Glycero-3-Phosphate (“DLPA”), 1-Palmitoyl-2-Oleoyl-sn-Glycero-3-Phosphate (“POPA”), 1-Palmitoyl-2-Oleoyl-sn-Glycero-3-Phosphocholine (“POPC”) and 1-Palmitoyl-2-Oleoyl-sn-Glycero-3-[Phospho-L-Serine] (“POPS”) were from Avanti Polar Lipids (Alabaster, Ala.). Epidermal growth factor (“EGF”) was from Invitrogen (Carlsbad, Calif.). Glutathione Sepharose 4B was from Amersham Biosciences (Piscataway, N.J.), and Ni-NTA resin was from Pierce (Rockford, Ill.).

Antibodies

The anti-T7 antibody was from Novagen (Madison, Wis.). The anti-phosphotyrosine (4G10), anti-EGFR, anti-HA, and anti-Ras antibodies were from Upstate (Lake Placid, N.Y.). HRP-conjugated anti-mouse was from MP Biomedicals (Solon, Ohio). FITC-conjugated goat anti-mouse antibody was from Sigma-Aldrich (St. Louis, Mo.). Anti-Ras antibody (Clone Ras10) was bought from Calbiochem (San Diego, Calif.). The anti-PLD2 antibody was a gift from Dr. Yoshinori Nozawa (Gifu International Institute of Biotechnology).

Cell Culture and Transfection

HeLa and COS-1 cells were cultured in DMEM supplemented with 10% or 5% fetal bovine serum (“FBS”) (Invitrogen, Carlsbad, Calif.), respectively. NIH 3T3 cells were grown in DMEM supplemented with 10% calf serum. The cells were maintained in 5% CO₂ at 37° C. Transient transfections were performed with the Fugene 6 transfection reagent from Roche (Indianapolis, Ind.) according to the manufecturer's instructions.

Plasmids

HA-tagged H-Ras [Ras(amino acids 1-189)], wild type human Sos1 [Sos (amino acids 1-1333)] (SEQ ID NO:1), and Sos-ΔC (amino acids 1-1049) (SEQ ID NO:3) were previously described (Corbalan-Garcia et al., “Regulation of Sos Activity by Intramolecular Interactions,” Mol. Cell. Biol. 18:880-886 (1998), which is hereby incorporated by reference in its entirety). His- and T7-tagged Sos-PH were previously described (Chen et al., “The Role of the PH Domain in the Signal-dependent Membrane Targeting of Sos,” EMBO J. 16:1351-1359 (1997), which is hereby incorporated by reference in its entirety). GFP-tagged Sos-PH domain was generated by cloning the sequence corresponding to Sos amino acids 422-551 (SEQ ID NO:4) into pEGFP3 mammalian expression vector. Sos mutants were generated using PCR-based mutagenesis and constructs were verified by DNA sequencing. GST-Raf-1-RBD was described previously (Boykevisch, et al., “Regulation of Ras Signaling Dynamics by Sos-mediated Positive Feedback,” Curr. Biol. 16:2173-2179 (2006), which is hereby incorporated by reference in its entirety). GFP-PLD2, GFP-PLD2K758R, pCGN-PLD2, shRNA control plasmid, and shRNA plasmid for PLD2 knockdown constructs were described previously (Sung et al., “Mutagenesis of Phospholipase D Defines a Superfamily Including a Trans-golgi Viral Protein Required for Poxvirus Pathogenicity,” EMBO J. 16:4519-4530 (1997); Du et al., “Phospholipase D2 Localizes to the Plasma Membrane and Regulates Angiotensin II Receptor Endocytosis,” Mol. Biol. Cell. 15:1024-1030 (2004), which are hereby incorporated by reference in their entirety). The PLD2 constructs in the pMX retroviral vector were provided by Dr. Ling Zheng (Washington University, WA).

Lipid Binding Assays

The expression and purification of Sos-PH and the generation of lipid vesicles were described previously (Chen et al., “The Role of the PH Domain in the Signal-dependent Membrane Targeting of Sos,” EMBO J. 16:1351-1359 (1997), which is hereby incorporated by reference in its entirety). The binding reaction was initiated by adding purified Sos-PH to the lipid vesicles followed by the incubation of the lipid-protein mixture at room temperature for 30 min. At the end of the incubation period, the solution was centrifuged at 100,000 g for 45 minutes at 4° C. The supernatant was removed immediately, the pellet fraction was washed, and the bound protein was eluted by incubation with SDS-PAGE loading buffer. Binding affinities were determined by adding increasing amounts of Sos-PH to a fixed concentration of lipid vesicles. The vesicles pelleted and protein concentrations in both the supernatant and in the pellet fraction were determined by Micro BCA protein assay (Pierce, Rockford, Ill.). Dissociation constants were calculated from the Scatchard equation, Y/[Sos-PH]_{f}=Ka (1−Y), where [Sos-PH]_{f} is the fraction of free PH proteins in solution after centrifugation, Ka is the equilibrium association constant, and Y is the fraction of macromolecule sites occupied by the ligand. [Sos-PH]_{b}/[Lipid]·[Sos-PH]_{b} is the fraction of the Sos-PH bound to the lipid vesicles after centrifugation. Lipid concentration was considered to be half of the total, based on the assumption that only the outer leaflet of the vesicle is available for protein binding. The reciprocal of these numbers can be taken as an apparent dissociation constant.

Ras Activation Assay

The levels of Ras-GTP were determined by the GST-RBD pull down assay as described previously (Boykevisch et al., “Regulation of Ras Signaling Dynamics by Sos-mediated Positive Feedback,” Curr. Biol. 16:2173-2179 (2006), which is hereby incorporated by reference in its entirety).

Fluorescence Microscopy

Transfected cells grown on glass coverslips were fixed and processed for indirect immunofluorescence and direct fluorescence as described previously (Zheng et al., “The Solution Structure of the Pleckstrin Homology Domain of Human SOS1. A Possible Structural Role for the Sequential Association of Diffuse B Cell Lymphoma and Pleckstrin Homology Domains,” J. Biol. Chem. 272: 30340-30344 (1997); Du et al., “Phospholipase D2 Localizes to the Plasma Membrane and Regulates Angiotensin II Receptor Endocytosis,” Mol. Biol. Cell. 15:1024-1030 (2004), which are hereby incorporated by reference in their entirety). Images were acquired with a Zeiss Axiovert 200M microscope equipped with an ApoTome system. Z-stacks were processed with a nearest neighbor estimation deconvolution algorithm.

PLD2 Silencing

HeLa cells were transfected with short hairpin RNAs directed to PLD2 or Luciferase (control). The targeting sequences have been described previously (Du et al., “Phospholipase D2 Localizes to the Plasma Membrane and Regulates Angiotensin II Receptor Endocytosis,” Mol. Biol. Cell 15:1024-1030 (2004), which is hereby incorporated by reference in its entirety). After 16 hr of expression, the media was replaced with fresh media supplemented with 10 μg/ml Blasticidin (Invitrogen, Carlsbad, Calif.) and transfected cells were selected for 72 hours.

NIH3T3 Foci Formation Assay

NIH 3T3 cells were first infected with retroviruses expressing either the control or PLD2 proteins. The retrovirus-infected cells were selected by blasticidin (10 ng/ml) for 4 days. The cells were then transfected with 0.2 ng of H-Ras, 1 ng of Sos-ΔC, or a Sos-ΔC-HR/EE mutant and maintained in DMEM supplemented with 5% calf serum for 14 days with a medium change every 3 days.

Lipid Micelles Preparation

PA-based lipid micelles were obtained by drying a DLPA/chloroform solution under argon gas. The lipid powder was resuspended in DMEM and the solution was sonicated for 1 minute, snap-freezed in liquid nitrogen, and thawed in a 37° C. incubator. This cycle was repeated at least 8 times until the lipid mixture became semi-transparent.

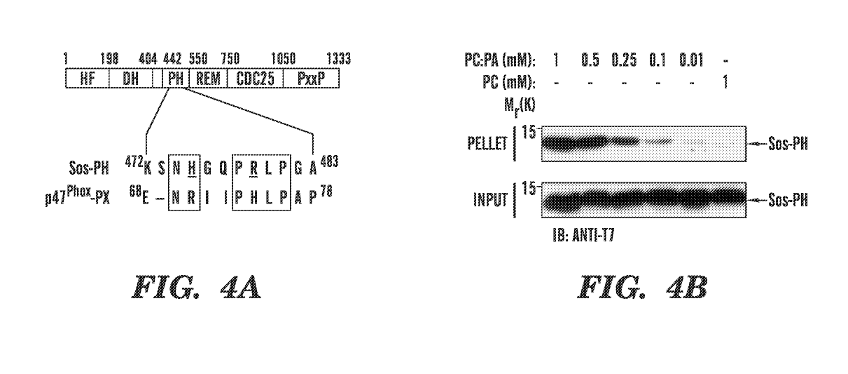

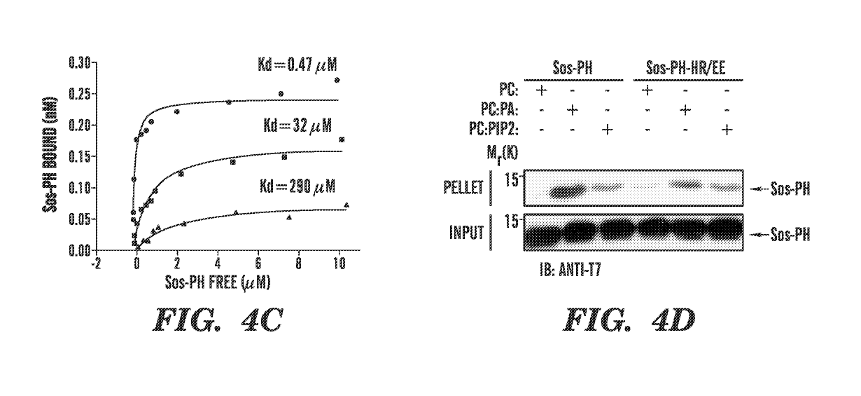

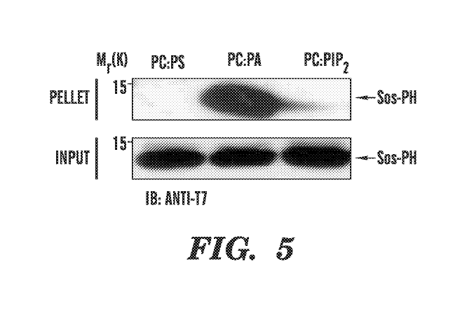

Inspection of the Sos-PH sequence unexpectedly revealed a short region of high similarity with the PX domain of the NADPH oxidase component p47^{phox} (p47^{phox}-PX, FIG. 4A (SEQ ID NO:5)). The conserved region contains basic amino acids that have been implicated in phosphatidic acid binding to p47^{phox}-PX (Karathanassis et al., “Binding of the PX Domain of p47(phox) to Phosphatidylinositol 3,4-bisphosphate and Phosphatidic Acid is Masked by an Intramolecular Interaction,” EMBO J. 21:5057-5068 (2002), which is hereby incorporated by reference in its entirety). PA is a negatively-charged phospholipid that can function as a lipid anchor via direct binding to positively-charged sites on effector proteins (Stace et al., “Phosphatidic Acid- and Phosphatidylserine-binding Proteins,” Biochim. Biophys. Acta 1761:913-926 (2006), which is hereby incorporated by reference in its entirety). Several signaling molecules, including the serine/threonine kinase Raf-1 and the NADPH oxidase component p47^{phox} have been shown to depend on PA binding for membrane translocation (Rizzo et al., “Phospholipase D and Its Product, Phosphatidic Acid, Mediate Agonist-dependent Raf-1 Translocation to the Plasma Membrane and the Activation of the Mitogen-activated Protein Kinase Pathway,” J. Biol. Chem. 274:1131-1139 (1999); Park, “Phosphatidic Acid-induced Translocation of Cytosolic Components in a Cell-free System of NADPH Oxidase: Mechanism of Activation and Effect of Diacylglycerol,” Biochem. Biophys. Res. Commun. 229:758-763 (1996), which are hereby incorporated by reference in their entirety). These observations raise the issue of whether Sos-PH interacts with PA in a functionally significant manner.

The capacity of Sos-PH to bind directly to PA was examined using a lipid vesicle sedimentation assay (Rebecchi et al., “Phosphoinositide-specific Phospholipase C-delta 1: Effect of Monolayer Surface Pressure and Electrostatic Surface Potentials on Activity,” Biochemistry 31:12748-12753 (1992), which is hereby incorporated by reference in its entirety). Purified Sos-PH was incubated with lipid vesicles containing PC mixed with PA, PS, or PIP₂, at ratios reported in the literature for similar binding assays (Karathanassis et al., “Binding of the PX Domain of p47(phox) to Phosphatidylinositol 3,4-bisphosphate and Phosphatidic Acid is Masked by an Intramolecular Interaction,” EMBO J. 21:5057-5068 (2002); Rebecchi et al., “Phosphoinositide-specific Phospholipase C-delta 1: Effect of Monolayer Surface Pressure and Electrostatic Surface Potentials on Activity,” Biochemistry 31:12748-12753 (1992); Fang et al., “Phosphatidic Acid-mediated Mitogenic Activation of mTOR Signaling,” Science 294:1942-1945 (2001), which are hereby incorporated by reference in their entirety). The vesicles were then pelleted and analyzed by Western blotting for the amount of Sos-PH pulled down. As illustrated in FIG. 4B, Sos-PH bound to the PA-containing vesicles in a concentration-dependent manner with detectable binding being observed at a concentration of 0.001 mM PA (0.01 mM total lipid). In contrast, no binding was observed in the presence of vesicles composed solely of PC even at 1000-times higher concentration (FIG. 4B) or to PS-containing vesicles, another acidic phospholipid (FIG. 5), consistent with earlier findings (Zheng et al., “The Solution Structure of the Pleckstrin Homology Domain of Human SOS1. A Possible Structural Role for the Sequential Association of Diffuse B Cell Lymphoma and Pleckstrin Homology Domains,” J. Biol. Chem. 272:30340-30344 (1997), which is hereby incorporated by reference in its entirety). Sos-PH also binds to PIP₂-containing vesicles (FIG. 4D).

To confirm the relevance of the hypothetical PA-binding motif (FIG. 4A), the two positively-charged Sos residues, H475 and R479 (FIG. 4A, Sos-PH, KSNHGQPRLPGA (SEQ ID NO:6)), were mutated to glutamic acid (Sos-PH-HR/EE) (KSNEGQPELPGA (SEQ ID NO:7)) and the effect of these mutations on PA binding was examined by mixing PA-containing vesicles with increasing amounts of wild-type Sos-PH or Sos-PH-HR/EE (FIG. 4C). Sos-PH bound to the lipid vesicles with an apparent dissociation constant (Kd) of 0.47 μM, which was 1000-times stronger than the binding observed for vesicles containing only PC (290 μM). In comparison to wild-type Sos-PH, the binding affinity of the Sos-PH-HR/EE mutant for PA-containing vesicles was reduced by approximately 80-fold (Kd=32 μM). These results identify residues H475 and R479 as being critical for the high affinity interaction between Sos-PH and PA, although it is possible that other residues make minor contributions. Of relevance, substitution of glutamic acid for R70 in p47^{phox}-PX (FIG. 4A) similarly leads to a substantial decrease in PA binding affinity (Karathanassis et al., “Binding of the PX Domain of p47(phox) to Phosphatidylinositol 3,4-bisphosphate and Phosphatidic Acid is Masked by an Intramolecular Interaction. EMBO J. 21:5057-5068 (2002), which is hereby incorporated by reference in its entirety). The lipid-binding defect exhibited by Sos-PH-HR/EE was specific for PA in that no effect was observed on the binding of PIP₂ (FIG. 4D), suggesting that PA and PIP₂ bind to distinct determinants on Sos-PH. It should be noted that the proposed PA binding site on Sos-PH and the identified PA binding site on p47^{phox}-PX occur in structurally distinct regions within their respective domains.

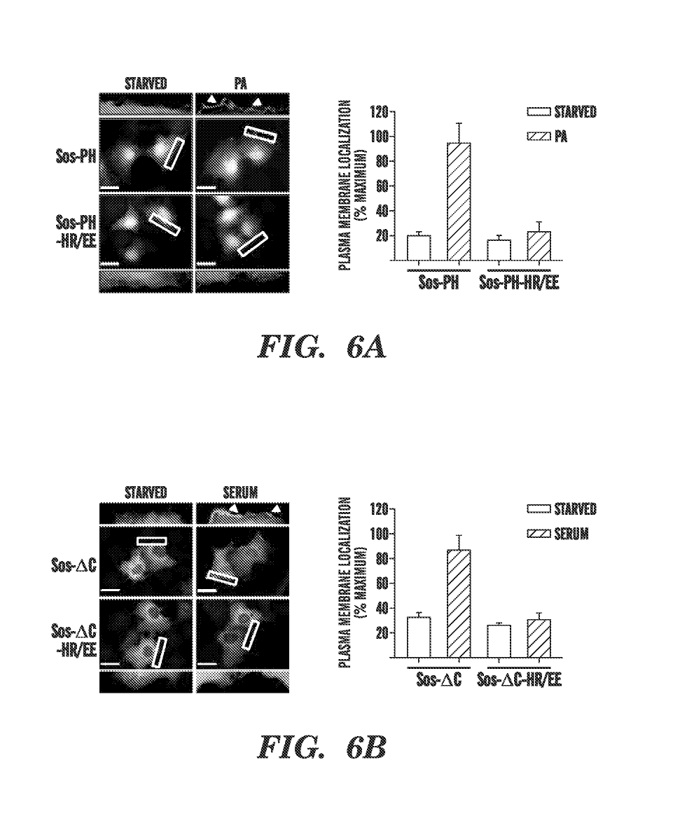

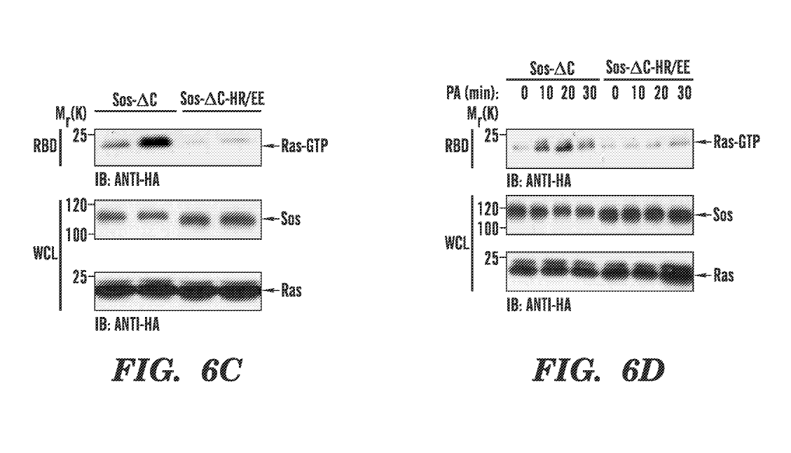

To determine whether PA binding is required for the membrane targeting of Sos-PH, GFP-fusion constructs of Sos-PH and Sos-PH-HR/EE were transiently transfected into COS-1 cells. The cells were treated with a membrane-permeable form of PA and the subcellular distribution of the GFP-tagged proteins was subsequently analyzed by fluorescence microscopy. In serum-deprived cells, Sos-PH localized predominantly to the nucleus and cytoplasm. The addition of PA stimulated Sos-PH translocation to the plasma membrane, as evident from the appearance of a rim of fluorescence at the cell periphery (FIG. 6A, arrowheads). In contrast, PA failed to induce plasma membrane recruitment of Sos-PH-HR/EE, indicating that the binding of PA to Sos-PH is necessary and sufficient for Sos-PH membrane translocation. Sos-PH-HR/EE was also defective in serum-induced plasma membrane translocation, suggesting a role for PA in mediating growth factor-dependent Sos-PH recruitment.

To verify that the observed role for PA binding is not restricted to the isolated Sos PH domain, the serum-induced translocation of epitope-tagged wild-type and HR/EE Sos constructs was examined by indirect immunofluorescence. To eliminate the contribution of Grb2-mediated plasma membrane recruitment, Sos truncation mutants lacking the C-terminal region (residues 1050-1333, FIG. 4A), hereafter referred to as SosΔC, were employed. In agreement with an earlier report (Wang, et al., “The Grb2 Binding Domain of mSos1 is not Required for Downstream Signal Transduction,” Nat. Genet. 10:294-300 (1995), which is hereby incorporated by reference in its entirety), Grb2 binding was not essential for the membrane targeting of Sos, as indicated by the capacity of SosΔC to undergo serum-induced membrane translocation (FIG. 6B). In contrast, a SosΔC construct mutated to eliminate PA binding (SosAC-HR/EE) was deficient in serum-induced plasma membrane targeting, confirming the critical role of PA binding in PH domain-mediated recruitment of Sos to the membrane.

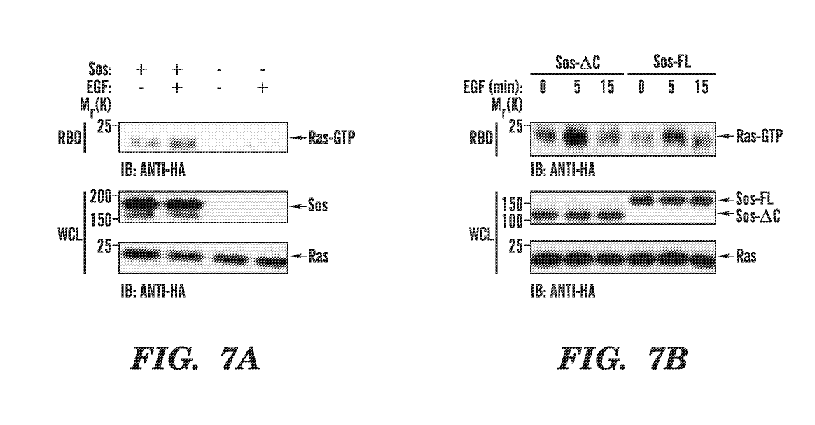

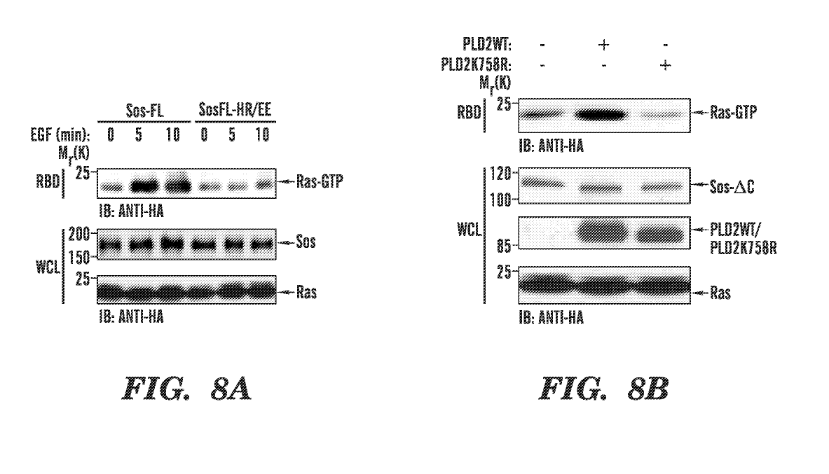

Because ligand-induced membrane recruitment of Sos is required for Ras activation, it was examined whether the binding of PA to Sos is critical for this process. COS-1 cells were co-transfected with differentially-tagged Sos and H-Ras (Ras) expression vectors, and Ras activation was monitored using the Raf-1 Ras Binding Domain (“RBD”) pull-down assay (de Rooij et al., “Minimal Ras-binding Domain of Raf1 can be Used as an Activation-specific Probe for Ras,” Oncogene 14:623-625 (1997), which is hereby incorporated by reference in its entirety). The relative expression levels of Ras and Sos were experimentally adjusted to ensure that the contribution of endogenous Sos to the Ras activation signal was negligible (FIG. 7A). As in the localization study described above, the SosΔC truncation mutant was used to rule out activation that might ensue from a Grb2-Sos interaction. In agreement with earlier observations (Wang et al., “The Grb2 Binding Domain of mSos1 is not Required for Downstream Signal Transduction,” Nat. Genet. 10:294-300 (1995); Karlovich et al., “In Vivo Functional Analysis of the Ras Exchange Factor Son of Sevenless,” Science 268:576-579 (1995), which is hereby incorporated by reference in its entirety), the ligand-induced activation of Ras by Sos was not compromised by the elimination of the Grb2 binding (FIG. 6C, FIG. 7B). These results are mechanistically consistent with the conclusion that Sos-PH contains necessary and sufficient membrane translocation determinants. In contrast, SosΔC-HR/EE failed to activate Ras in response to EGF stimulation (FIG. 6C). The dependence of Sos-mediated Ras activation on PA binding was also displayed by full-length Sos (FIG. 8A), indicating that the interaction of PA with the PH domain of Sos is an obligatory step in the process that couples EGF receptor stimulation to Ras activation. In support of this model, treatment of cells with membrane-permeable PA sufficed to induce Ras activation by SosΔC, but did not increase the level of active Ras in cells expressing the PA binding-deficient mutant SosΔC-HR/EE (FIG. 6D). Since the substitution of HR for EE in Sos-PH did not alter the intrinsic catalytic activity of Sos, it was concluded that the regulatory function of PA is to promote the membrane association of Sos, which in turn triggers Ras activation. The extent to which additional protein-protein or protein-lipid interactions may contribute to this targeting mechanism remains to be determined.

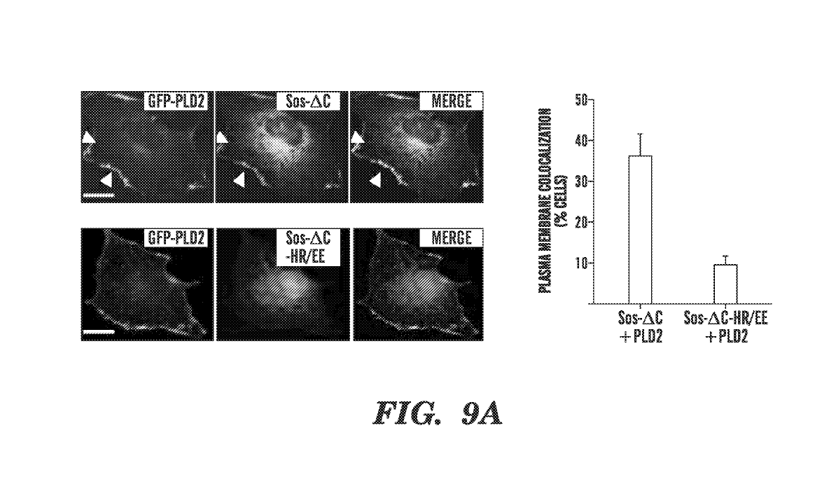

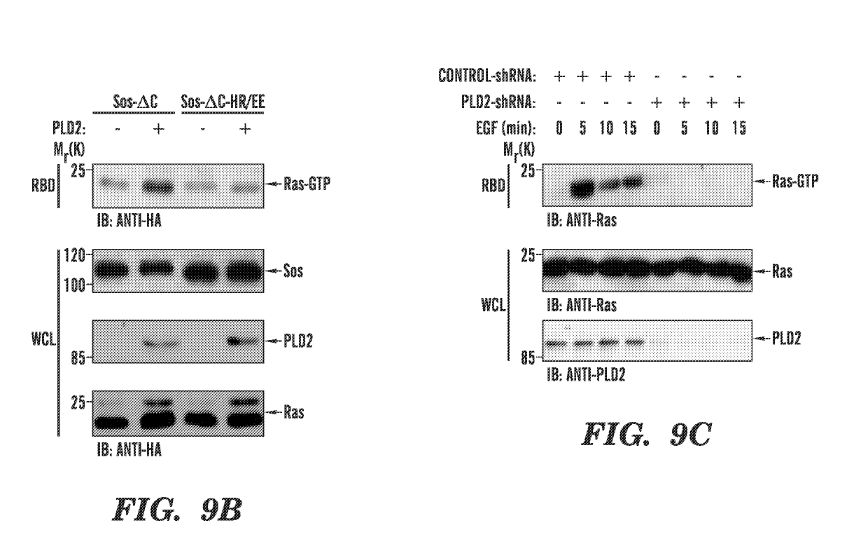

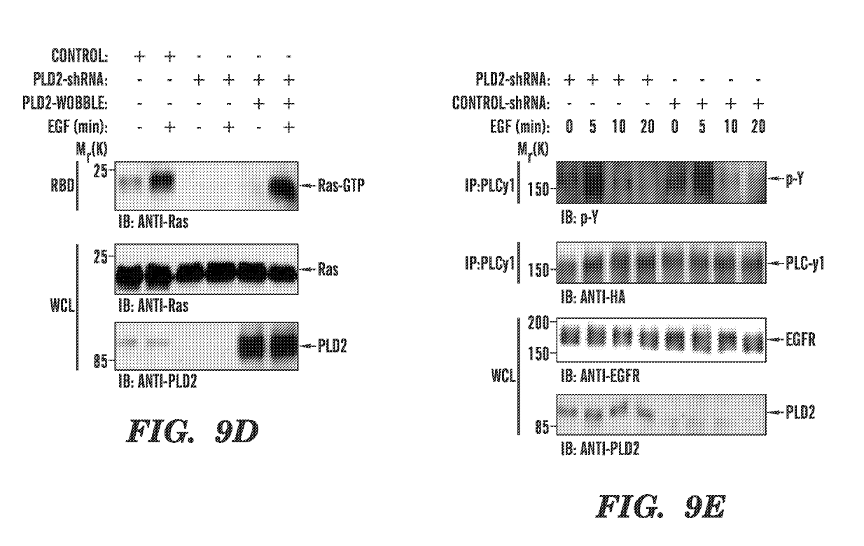

Both biosynthetic and signaling pathways can lead to the production of PA. Among the latter, the best characterized routes involve the action of lysophosphatidic acid acetyltransferases (“LPAATs”) that acetylate lysophosphatidic acid to generate PA, diacylglycerol (“DAG”) kinases that accomplish this by phosphorylating DAG, and phospholipase Ds (“PLDs”), which create PA from the hydrolysis of PC (Wang et al., “Signaling Functions of Phosphatidic Acid,” Prog. Lipid Res. 45:250-278 (2006), which is hereby incorporated by reference in its entirety). The isoform PLD2 was previously described as localizing to the plasma membrane (Colley et al., “Phospholipase D2, a Distinct Phospholipase D Isoform with Novel Regulatory Properties that Provokes Cytoskeletal Reorganization,” Curr. Biol. 7:191-201 (1997), which is hereby incorporated by reference in its entirety). In addition, PLD2 has been reported to complex with the EGF receptor and to be activated by EGF-signaling (Slaaby et al., “PLD2 Complexes with the EGF Receptor and Undergoes Tyrosine Phosphorylation at a Single Site upon Agonist Stimulation,” J. Biol. Chem. 273:33722-33727 (1998), which is hereby incorporated by reference in its entirety). Accordingly, it was investigated whether the presumed EGF-triggered increases in PA and the resulting Sos translocation and Ras activation could be mediated by PLD2. Co-expression of GFP-PLD2 and wild-type Sos-ΔC in COS-1 cells revealed that the provision of elevated levels of PLD2, which localizes to the plasma membrane (FIG. 9A) and exhibits substantial basal activity when overexpressed (Colley et al., “Phospholipase D2, a Distinct Phospholipase D Isoform with Novel Regulatory Properties that Provokes Cytoskeletal Reorganization,” Curr. Biol. 7:191-201 (1997), which is hereby incorporated by reference in its entirety), sufficed to drive the translocation of wild-type Sos-ΔC to the plasma membrane (FIG. 9A, top row). In contrast, the PA binding-deficient Sos mutant (Sos-AC-HR/EE), remained cytosolic when co-expressed with PLD2 (FIG. 9A, bottom row). Ras activation was examined similarly and found to be stimulated by the co-expression of PLD2 with Sos-ΔC but not Sos-AC-HR/EE (FIG. 9B). This stimulatory effect was abolished upon the expression of a catalytically-inactive mutant form of PLD2 (Sung et al., “Mutagenesis of Phospholipase D Defines a Superfamily Including a Trans-golgi Viral Protein Required for Poxvirus Pathogenicity,” EMBO J. 16:4519-4530 (1997), which is hereby incorporated by reference in its entirety) (FIG. 8B). Taken together, these findings show that the induction of Sos recruitment and Ras activation in response to elevated PLD2 signaling can be attributed to the binding of the product of PLD2's activity, PA, to the PH domain of Sos.

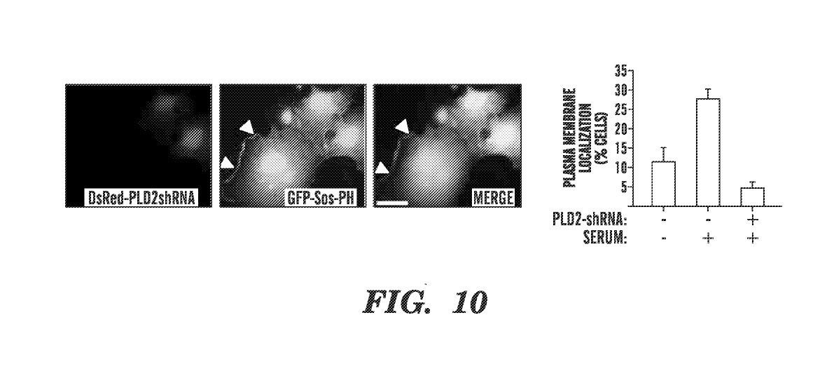

Overexpression phenotypes can be misleading with respect to roles mediated by proteins at their endogenous levels of expression. To examine whether endogenous PLD2 is required for ligand-induced Ras activation, an RNAi approach was employed that has been previously used successfully for PLD2 (Du et al., “Phospholipase D2 Localizes to the Plasma Membrane and Regulates Angiotensin II Receptor Endocytosis,” Mol. Biol. Cell 15:1024-1030 (2004), which is hereby incorporated by reference in its entirety). PLD2 knockdown by small hairpin RNA (“shRNA”), which was approximately 90% effective, dramatically blocked EGF-stimulated Ras activation in comparison to control cells transfected with the shRNA directed against Luciferase (FIG. 9C). Similar to the effects observed for Ras activation, PLD2 knock-down inhibited the serum-induced translocation of Sos-PH (FIG. 10). To confirm that the loss of Ras activation was due specifically to PLD2 knock-down rather than to a stress response or off-target cleavage of unrelated mRNAs, PLD2 shRNA-expressing cells were transfected with a PLD2 “rescue” expression plasmid mutated at wobble codons within the shRNA-targeted region to render it resistant to RNAi-mediated cleavage. Expression of the wobble-mutated PLD2 cDNA restored the ability of EGF to induce Ras activation (FIG. 9D), confirming the role for PLD2 as a critical intermediate in this activation process. To ensure that observed effects did not reflect changes in the activation capacity of the EGFR due to PLD2 knock-down, the tyrosine phosphorylation status of phospholipase C (PLC)-γ1 in PLD2 knock-down cells stimulated with EGF was examined. In this context, it is important to note that the EGFR-mediated signaling pathway that leads to the activation and phosphorylation of PLC-yl does not depend on Sos recruitment and Ras activation (Bogdan et al., “Epidermal Growth Factor Receptor Signaling,” Curr. Biol. 11:R292-295 (2001), which is hereby incorporated by reference in its entirety). No differences were observed in the levels of PLC-γ1 tyrosine phosphorylation between cells expressing PLD2 shRNA and cells expressing control shRNA (FIG. 9E). This indicates that the EGF receptor undergoes normal activation in the absence of PLD2, and thus that the signaling defect in Ras activation lies downstream of this event. Taken together, these findings show that PLD2 is necessary and sufficient to trigger Sos recruitment to the plasma membrane and Ras activation in response to growth factor stimulation. It has been shown that PLD1 is a downstream target of Ras and can serve as an activator of PLD2 (Foster et al., “Phospholipase D in Cell Proliferation and Cancer,” Mol. Cancer. Res. 1:789-800 (2003); Luo et al., “RalA Interacts Directly with the Arf-responsive, PIP2-dependent Phospholipase D1,” Biochem. Biophys. Res. Commun. 235:854-859 (1997), which are hereby incorporated by reference in their entirety). Hence Ras activation may be additionally controlled by a positive feedback loop generated through PLD-dependent production of PA.

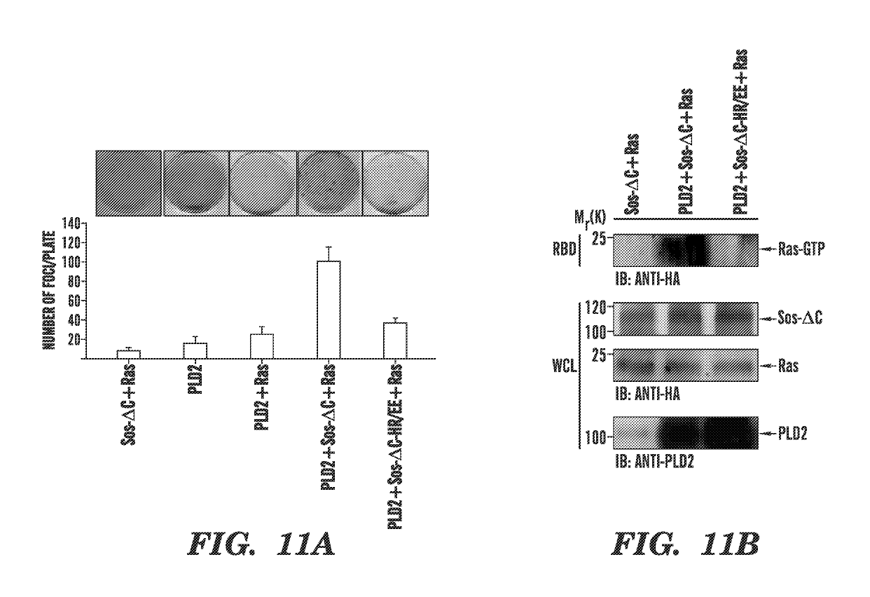

Hyperactivation of Ras and its downstream effector pathways has been causally linked to acquisition of the transformed phenotype (Malumbres et al., “RAS Oncogenes: The First 30 Years,” Nat. Rev. Cancer 3:459-465 (2003), which is hereby incorporated by reference in its entirety). In view of the finding that PLD2 overexpression promotes Ras activation, it was next examined whether the pathway that leads to this activation, namely PLD2-mediated Sos recruitment, contributes to the transforming potential of Ras. NIH3T3 cells were co-transfected with Ras and SosAC expression plasmids with or without PLD2, and transformation was scored by foci formation. Western blot analysis confirmed that the expression level of each protein was equal in the different experimental conditions (FIG. 11B). The expression of Ras in the presence of SosΔC gave rise to a very small number of foci (FIG. 11A). Likewise, the expression of PLD2 in itself led only to a weak foci-forming activity. In contrast, the combination of Ras, SosΔC, and PLD2 resulted in a marked increase in foci number, and this synergistic effect was abolished when the PA binding-deficient Sos mutant SosΔC-HR/EE was used instead. Finally, the PLD2-mediated enhancement of the transforming capacity of Ras was associated with a PA binding-dependent increase in the level of active Ras (FIG. 11B). Both PLD overexpression and elevated PLD activity have been noted in several human cancers (Malumbres et al., “RAS Oncogenes: The First 30 Years,” Nat. Rev. Cancer 3:459-465 (2003); Fiucci et al., “Changes in Phospholipase D Isoform Activity and Expression in Multidrug-resistant Human Cancer Cells,” Int. J. Cancer 85:882-888 (2000); Welsh et al., “Increased Phospholipase D Activity in Multidrug Resistant Breast Cancer Cells,” Biochem. Biophys. Res. Commun. 202:211-217 (1994), which is hereby incorporated by reference in its entirety). These findings raise the possibility that in these settings, unrestrained cell growth may result, at least in part, from PLD-driven excessive Ras signaling.

The spatial alignment of Sos and Ras in the plasma membrane is crucial for the coupling of receptor stimulation to Ras activation. While the role of Grb2-Sos interaction in promoting the ligand-dependent recruitment of Sos to activated receptors is well established, the identification of Sos alleles that are defective in Grb2-binding but retain the capacity to activate Ras argues for the existence of additional determinants that can mediate the membrane targeting of Sos (Wang et al., “The Grb2 Binding Domain of mSos1 is not Required for Downstream Signal Transduction,” Nat. Genet. 10:294-300 (1995); Karlovich et al., “In Vivo Functional Analysis of the Ras Exchange Factor Son of Sevenless,” Science 268:576-579 (1995); McCollam et al., “Functional Roles for the Pleckstrin and Dbl Homology Regions in the Ras Exchange Factor Son-of-sevenless,” J. Biol. Chem. 270:15954-15957 (1995); Qian et al., “N Terminus of Sos1 Ras Exchange Factor: Critical Roles for the Dbl and Pleckstrin Homology Domains,” Mol. Cell. Biol. 18:771-778 (1998), which are hereby incorporated by reference in their entirety). There is a Grb2-independent mechanism for Sos-mediated Ras activation involving the PA-dependent tethering of Sos to the plasma membrane. Since PA is produced by the activation of a wide spectrum of cell surface receptors, this mechanism is likely to play a central role in the coupling of extracellular signals to Ras activation. The relative contribution of Grb2- and PA-mediated membrane targeting mechanisms to Sos function remains to be established. It is possible that the two mechanisms are utilized in a mutually exclusive manner depending on the signaling context and the physiological setting. Alternatively, these two mechanisms may act in concert with the PA-mediated recruitment providing the principle driving force for plasma membrane anchoring and the Grb2-mediated binding to activated receptors serving to fine-tune the localization of Sos to a particular domain within the plasma membrane. In addition, as proposed earlier (Wang et al., “The Grb2 Binding Domain of mSos1 is not Required for Downstream Signal Transduction,” Nat. Genet. 10:294-300 (1995), which is hereby incorporated by reference in its entirety), the interaction of Sos with Grb2 may contribute to the stabilization of membrane-bound Sos in a catalytically active conformation possibly through the alleviation of C-terminal mediated auto-inhibition. Intriguingly, the plasma membrane translocation of the Ras effector Raf-1 has been also shown to be mediated by PA (Rizzo et al., “Phospholipase D and Its Product, Phosphatidic Acid, Mediate Agonist-dependent Raf-1 Translocation to the Plasma Membrane and the Activation of the Mitogen-activated Protein Kinase Pathway,” J. Biol. Chem. 274:1131-1139 (1999); Rizzo et al., “The Recruitment of Raf-1 to Membranes is Mediated by Direct Interaction with Phosphatidic Acid and is Independent of Association with Ras,” J. Biol. Chem. 275:23911-23918 (2000), which are hereby incorporated by reference in their entirety). Thus, the membrane recruitment function of PA could serve to bring into proximity an activator and an effector of Ras, thereby maximizing the efficiency of signal propagation. These studies implicate PLD2 as the primary source of the PA pool that is responsible for Sos translocation and Ras activation. Recent findings suggest a crucial role of PLD2-PAP-mediated production of DAG in RasGRP1-induced Ras activation at plasma membrane in T cells. Both studies invoke for the first time a role for PLD2 as a critical upstream regulator of Ras and underscore the importance of lipid-protein interactions in the spatio-temporal regulation of Ras signaling.

### Example 2

### Honokiol Suppresses Survival Signals Mediated by Ras-Dependent Phospholipase D Activity in Human Cancer Cells

Cells, Cell Culture Conditions

All human cancer cell lines used in this study were obtained from the American Type Culture Collection and were maintained in Dulbecco's modified Eagle's medium (“DMEM”) with 10% bovine calf serum (Sigma). Cos1 cells were maintained in DMEM and 5% fetal bovine serum as described previously (Zhao et al., “Phospholipase D2-generated Phosphatidic Acid Couples EGFR Stimulation to Ras Activation by Sos,” Nat. Cell Biol. 9:706-12 (2007), which is hereby incorporated by reference in its entirety).

Materials

[³H]-myristic acid was obtained from New England Nuclear. Precoated silica 60A thin layer chromatography plates were from Whatman. Honokiol was purified according to the method of Amblard et al. (Amblard et al., “Facile Purification of Honokiol and Its Antiviral and Cytotoxic Properties,” J. Med. Chem. 49:3426-7 (2006), which is hereby incorporated by reference in its entirety). Antibodies against HIF2a and hemaglutinin (“HA”) were obtained from Santa Cruz Biotechnology (Santa Cruz, Calif.). Antibodies raised against poly-(ADP-ribose) polymerase (“PARP”), actin, ribosomal subunit S6 kinase (“S6K”), phosphorylated-S6K, eukaryotic initiation factor 4E binding protein 1 (“4E-BP1”), phosphorylated-4EBP1 were purchased from Cell Signaling Technologies (Danvers, Mass.). Phosphatidylethanolamine, phosphatidylcholine, and phosphatidylinositol-4,5-bis-phosphate were purchased from Avanti Polar Lipids. [methyl-³H]-phosphatidylcholine was purchased from Perkin Elmer Life Sciences.

Plasmid Vectors

Vectors used were pcDNA3.1(−) (Invitrogen, Carlsbad, Calif.) and pcDNA3.1(−)-S17NRas, which was constructed by inserting the S17N Ras gene from pCMV-S17NRas (Clonetech, Mountain View, Calif.) using flanking EcoR1 and BamH1 sites. They were constructed by PCR amplification of the corresponding cDNAs and cloned into the EcoRI site of pcDNA3.1 (−) (Invitrogen). The ARF vectors pcDNA3.1-ARF1T31N and pcDNA3.1-ARF6T27N were described previously (D′ Souza-Schorey et al., “A Regulatory Role for ARF6 in Receptor-mediated Endocytosis,” Science 267:1175-8 (1995), which is hereby incorporated by reference in its entirety). The generation of the pCGN vectors expressing HA-tagged Sos and Ras were described previously (Corbalan-Garcia et al., “Regulation of Sos Activity by Intramolecular Interactions,” Mol. Cell. Biol. 18:880-6 (1998), which is hereby incorporated by reference in its entirety). The S28N RalA mutant cloned into pcDNA3.1(−) was described previously (Luo et al., “Functional Association Between RalA and Arf in Active Phospholipase D Complexes,” Proc. Natl. Acad. Sci. USA 95:3632-7 (1998), which is hereby incorporated by reference in its entirety).

Western Blot Analysis

Extraction of proteins from cultured cells and Western blot analysis of extracted proteins was performed using the ECL system (Amersham) as described previously (Chen et al., “Alternative Phospholipase D/mTOR Survival Signal in Human Breast Cancer Cells,” Oncogene 24:672-9 (2005), which is hereby incorporated by reference in its entirety).

Ras Activation Assays

Cells were harvested and lysates were prepared and treated with the Ras binding domain of Raf1 (Pierce, Rockford, Ill.) according to the vendor's instructions. Ras-GTP bound to the Raf1 binding domain was recovered and subjected to Western blot analysis using a Pan-Ras antibody supplied with the Ras activation assay kit. For the Cos1 cell assays, an HA antibody was used to bind the ectopically-expressed Ras and Sos proteins.

Cell Viability and Apoptosis

Cell viability was determined by trypan blue exclusion. After various treatments, cells were harvested, washed, and treated with trypan blue at a concentration of 0.4% w/v. After 10 min, trypan blue uptake (dead cells) was determined by counting on a hemocytometer. Apoptosis was evaluated by examination of cleavage of the caspase 3 substrate PARP as described previously (Chen et al., “Alternative Phospholipase D/mTOR Survival Signal in Human Breast Cancer Cells,” Oncogene 24:672-9 (2005), which is hereby incorporated by reference in its entirety).

Phospholipase D Assays

For in vivo PLD activity, cells were plated in 60 mm culture dishes in DMEM with 10% serum at a density that would allow them to grow to about 80% confluence after 2 days, then cells were shifted to DMEM containing 0.5% or 10% serum as indicated in the figure legends overnight. Cells were then prelabeled for 4 h with [³H]-Myristate (3 μCi, 40 Ci/mmol) in 3 ml of medium. PLD catalyzed transphosphatidylation in the presence of 0.8% 1-BtOH, and the extraction and characterization of lipids by thin layer chromatography was performed as described previously (Shen et al., “Phospholipase D Requirement for Receptor-mediated Endocytosis,” Mol. Cell. Biol. 21:595-602 (2001), which is hereby incorporated by reference in its entirety). For in vitro PLD activity, assays were performed with exogenous substrate as described previously (Henage et al., “Kinetic Analysis of a Mammalian Phospholipase D: Allosteric Modulation by Monomeric GTPases, Protein Kinase C, and Polyphosphoinositides,” J. Biol. Chem. 281:3408-17 (2006), which is hereby incorporated by reference in its entirety). Briefly, PLD activity was measured by the release of [methyl-³H] choline from [choline-methyl-³H]dipalmitoyl-phosphatidylcholine. 1-10 nM PLD was reconstituted with phospholipid vesicle substrates composed of 10 μM dipalmitoyl-phosphatidylcholine, 100 μM phosphatidylethanolamine, 6.2 μM phosphatidylinositol-4,5-bisphosphate, and 1.4 μM cholesterol. Assays were conducted for 30 min at 37° C. in 50 mM Hepes, pH 7.5, 80 mM KCl, 3 mM EGTA, 0.1 mM DTT, 3.6 mM MgCl₂, 3.6 mM CaCl₂, and 10 μM GTPγS. Reactions were stopped by the addition of trichloroacetic acid and bovine serum albumin. Free [methyl-³H] choline was separated from precipitated lipids and proteins by centrifugation and was analyzed by liquid scintillation counting. The counts present in the absence of added enzyme (i.e., background) were subtracted from other samples. ADP-ribosylation factor 1 (“Arf1”), PLD1, and PLD2 used in these assays were prepared as described previously (Henage et al., “Kinetic Analysis of a Mammalian Phospholipase D: Allosteric Modulation by Monomeric GTPases, Protein Kinase C, and Polyphosphoinositides,” J. Biol. Chem. 281:3408-17 (2006), which is hereby incorporated by reference in its entirety).

Stress-Induced PLD Activity in MDA-MB-231 Cells is Dependent on Ras

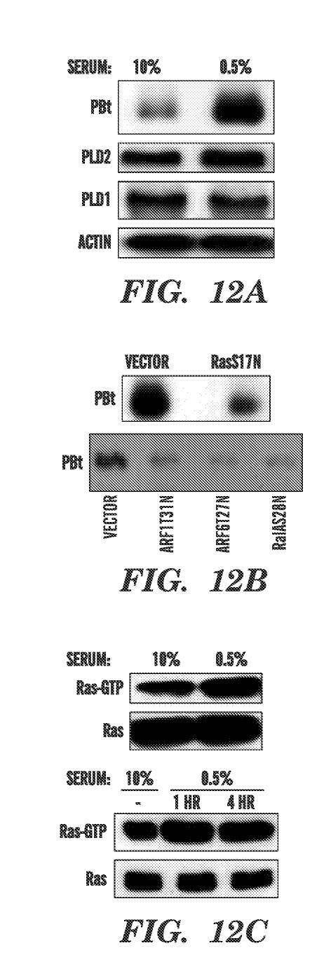

It was previously reported that serum withdrawal led to increased PLD activity in MDA-MB-231 cells (Zheng et al., “Phospholipase D Couples Survival and Migration Signals in Response to Stress in Human Breast Cancer Cells,” J. Biol. Chem. 281:15862-8 (2006), which is hereby incorporated by reference in its entirety). As shown in FIG. 12A, the increased PLD activity was not due to any changes in the levels of the PLD isoforms PLD1 and PLD2. This suggested that the increased PLD activity observed in response to the stress of serum withdrawal was due to increased activity, not increased protein levels. The regulation of PLD activity has been reported previously to involve a GTPase cascade of Ras and RalA (Foster et al., “Phospholipase D in Cell Proliferation and Cancer,” Mol. Cancer. Res. 1:789-800 (2003), which is hereby incorporated by reference in its entirety). RalA, which constitutively associates with PLD1 (Luo et al., “Ral Interacts Directly with the Arf-responsive PIP₂-dependent Phospholipase D1,” Biochem. Biophys. Res. Comm. 235:854-9 (1997), which is hereby incorporated by reference in its entirety), recruits ADP-ribosylation factor (“ARF”) GTPases into PLD complexes (Luo et al., “Functional Association between RalA and Arf in Active Phospholipase D Complexes,” Proc. Natl. Acad. Sci. USA 95:3632-7 (1998); Xu et al., “Elevated Phospholipase D Activity in H-Ras-, but not K-Ras-transformed Cells by the Synergistic Action of RalA and Arf6,” Mol. Cell. Biol. 23:645-64 (2003), which is hereby incorporated by reference in its entirety), where ARF actually stimulates the PLD activity of PLD1 (Brown et al., “ADP-ribosylation Factor, a Small GTP-dependent Regulatory Protein, Stimulates Phospholipase D Activity,” Cell 75:1137-44 (1993), which is hereby incorporated by reference in its entirety). The effect of dominant negative mutants for H-Ras, RalA, and ARF1 and ARF6, on the PLD activity in serum deprived MDA-MB-231 cells was examined. As shown in FIG. 12B, dominant negative mutants for H-Ras, RalA, and ARF all suppressed the stress-induced PLD activity in the MDA-MB-231 cells, indicating that Ras-RalA-PLD1 pathway (Foster et al., “Phospholipase D in Cell Proliferation and Cancer,” Mol. Cancer. Res. 1:789-800 (2003), which is hereby incorporated by reference in its entirety) is activated. The effect of serum withdrawal on Ras activation in the MDA-MB-231 cells were then examined. As shown in FIG. 12C, serum withdrawal led to an increase in GTP-bound Ras, while having no effect on total Ras levels. This effect could be detected within 1 hr (FIG. 12C). Since serum growth factors can also induce Ras activation, the surprising increase in Ras GTP binding observed upon serum withdrawal may actually be more significant than what is observed. The data in FIG. 12 reveal that the increased PLD activity observed in response to the stress of serum withdrawal is dependent upon both Ras and RalA, and that there is a rapid increase in Ras activation in MDA-MB-231 cells subjected to the stress of serum withdrawal.

Honokiol Suppresses Stress-Induced PLD Activity in MDA-MB-231 Cells

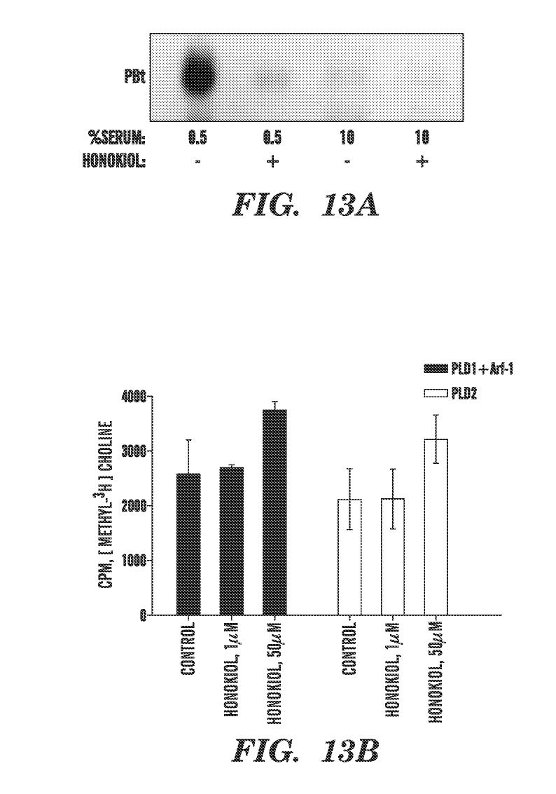

Honokiol was recently reported to suppress the growth of MDA-MB-231 human breast cancer cells in a mouse xenograft tumorigenesis assay (Wolf et al., “Honokiol, a Natural Biphenyl, Inhibits In Vitro and In Vivo Growth of Breast Cancer through Induction of Apoptosis and Cell Cycle Arrest,” Int. J. Oncol. 30:1529-37 (2007), which is hereby incorporated by reference in its entirety). It was reported previously that these cells depend upon PLD activity for their survival and their ability to migrate in culture (Chen et al., “Alternative Phospholipase D/mTOR Survival Signal in Human Breast Cancer Cells,” Oncogene 24:672-9 (2005); Zheng et al., “Phospholipase D Couples Survival and Migration Signals in Response to Stress in Human Breast Cancer Cells,” J. Biol. Chem. 281:15862-8 (2006); Zhong et al., “Phospholipase D Prevents Apoptosis In v-Src-transformed Rat Fibroblasts and MDA-MB-231 Breast Cancer Cells,” Biochem. Biophys. Res. Comm. 302:615-9 (2003), which is hereby incorporated by reference in its entirety). Therefore, it was examined whether honokiol had any effect on the highly elevated PLD activity observed in MDA-MB-231 cells subjected to serum withdrawal. MDA-MB-231 cells were placed in media containing either 10% or 0.5% serum for 18 hr at which time the PLD activity was evaluated in the presence and absence of honokiol for 4 hr. As shown in FIG. 13A, honokiol completely suppressed PLD activity in the cells in 0.5% serum while having little effect of the basal PLD activity in the cells maintained in 10% serum. These data indicate that honokiol specifically suppresses the PLD activity elevated in response to the stress of serum withdrawal, while having minimal effect on the basal PLD activity in the MDA-MB-231 cells.

Since honokiol suppressed the PLD activity elevated in response to the stress of serum withdrawal, the effect of honokiol on in vitro PLD activity was examined using purified recombinant PLD1 and PLD2 protein. As shown in FIG. 13B, honokiol had no significant effect upon the activity of either PLD1 or PLD2. ARF-1 is required for the in vitro activity of PLD1 and was included in the reaction with PLD1. Thus, the effect of honokiol is likely upstream of either PLD1 or PLD2 in the MDA-MB-231 cells and targets a regulatory mechanism for activating PLD in response to the stress of serum withdrawal.

Honokiol Suppresses Ras Activation

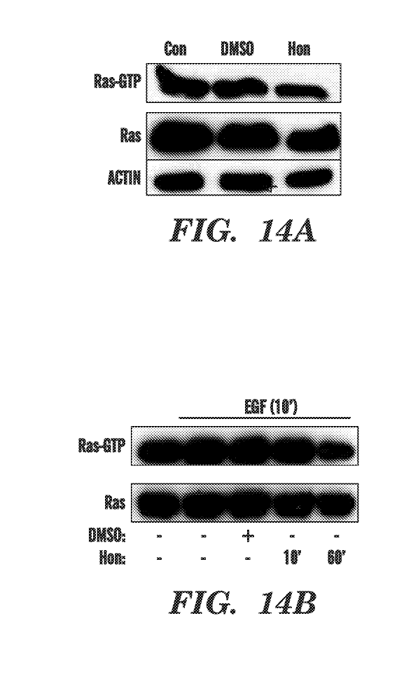

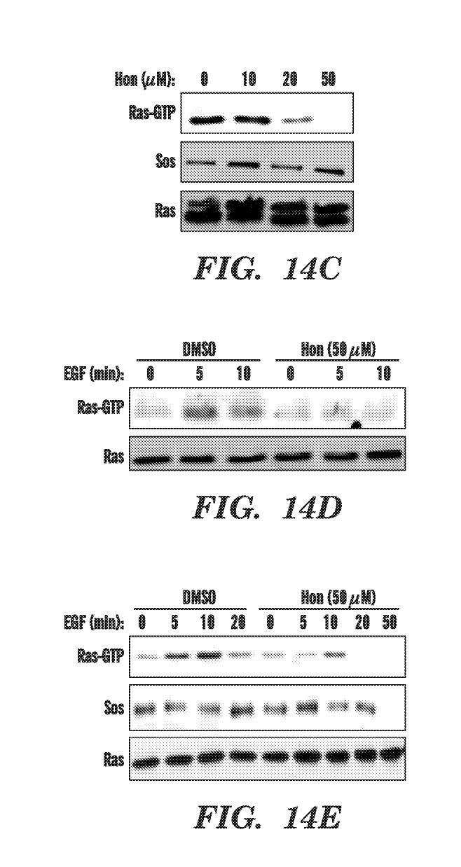

As indicated in FIG. 12, the PLD activity in MDA-MB-231 cells is dependent on Ras and RalA. Since honokiol suppressed the PLD activity in the MDA-MB-231 cells and this PLD activity was dependent upon Ras, the effect of honokiol on Ras activation was examined in the MDA-MB-231 cells. A “pull-down” assay was used that employs the Ras binding domain of Raf1, which recognizes GTP-bound Ras. As shown in FIG. 14A, honokiol suppressed the level of GTP-bound Ras. The effect of honokiol on Ras activation was small, but reproducible. This is likely due to the activated K-Ras present in these cells (Kozma et al., “The Human c-Kirsten Ras Gene is Activated by a Novel Mutation in Codon 13 in the Breast Carcinoma Cell Line MDA-MB231,” Nucleic Acids Res. 15:5963-71 (1987); Ogata et al., “Human Breast Cancer MDA-MB-231 Cells Fail to Express the Neurofibromin Protein, Lack Its Type I mRNA Isoform and Show Accumulation of P-MAPK and Activated Ras,” Cancer Leu. 172:159-64 (2001), which is hereby incorporated by reference in its entirety), which would give a high background of Ras GTP binding. MDA-MB-231 cells express high levels of the epidermal growth factor (“EGF”) receptor (Lev et al., “Dual Blockade of EGFR and ERK1/2 Phosphorylation Potentiates Growth Inhibition of Breast Cancer Cells,” Br. J. Cancer 91:795-802 (2004), which is hereby incorporated by reference in its entirety) and EGF stimulates both Ras activation and increased PLD activity (Shen et al., “Phospholipase D Requirement for Receptor-mediated Endocytosis,” Mol. Cell. Biol. 21:595-602 (2001); Lu et al., “Phospholipase D and RalA Cooperate with the EGF Receptor to Transform 3Y1 Rat Fibroblasts,” Mol. Cell. Biol. 20:462-7 (2000), which is hereby incorporated by reference in its entirety). The effect of honokiol on Ras activation in response to EGF in the MDA-MB-231 cells was therefore examined. As shown in FIG. 14B, honokiol also suppressed the Ras activation induced by EGF in the MDA-MB-231 cells.

To further establish that honokiol was suppressing Ras activation, the effect in Cos1 cells was examined where background Ras activation was lower. HA-tagged Ras and Sos were transiently transfected into the Cos1 cells with excess Sos to stimulate Ras activation as described previously (Boykevisch et al., “Regulation of Ras Signaling Dynamics by Sos-mediated Positive Feedback,” Curr. Biol. 16:2173-9 (2006), which is hereby incorporated by reference in its entirety). 24 hr later, the cells were then shifted to serum-free conditions in the presence of increasing concentrations of honokiol and the levels of Ras, Sos, and GTP-bound Ras was evaluated. As shown in FIG. 14C, honokiol strongly suppressed Ras-GTP levels at concentrations of 20 μM and higher. The effect of honokiol on the ability of EGF to induce Ras activation in Cos1 cells was also examined. As shown in FIG. 14D, honokiol suppressed the ability of EGF to increase the level of GTP-bound Ras. This experiment used endogenous Sos and ectopically expressed Ras. As shown in FIG. 14E, the effect of honokiol was more pronounced when ectopically expressed Sos was introduced, indicating that Ras activation by Sos was affected. These data indicate that honokiol suppresses Ras activation.

Honokiol Suppresses Downstream Targets of PLD Survival Signals

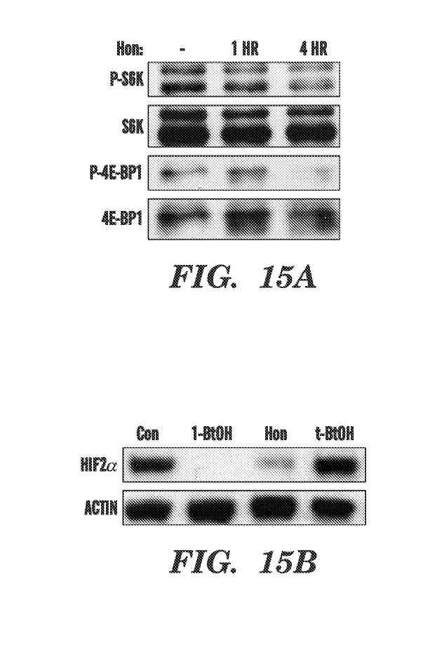

Elevated PLD activity in MDA-MB-231 cells has been reported to activate mTOR (Chen et al., “Alternative Phospholipase D/mTOR Survival Signal in Human Breast Cancer Cells,” Oncogene 24:672-9 (2005); Chen et al., “Phospholipase D Confers Rapamycin Resistance in Human Breast Cancer Cells,” Oncogene 22:3937-42 (2003), which are hereby incorporated by reference in their entirety), which has been correlated with survival signals in human cancer cells (Sawyers et al., “Will mTOR Inhibitors Make it as Cancer Drugs?” Cancer Cell 4:343-8 (2003), which is hereby incorporated by reference in its entirety). Elevated expression of PLD was also shown to lead to increased phosphorylation of the mTOR substrates ribosomal subunit S6 kinase (“S6K”) and eukaryotic initiation factor 4E binding protein 1 (“4E-BP1”) (Hui et al., “Phospholipase D Elevates the Level of MDM2 and Suppresses DNA Damage-induced Increases in p53,” Mol. Cell. Biol. 24:5677-88 (2004); Chen et al., “Phospholipase D Confers Rapamycin Resistance in Human Breast Cancer Cells,” Oncogene 22:3937-42 (2003), which are hereby incorporated by reference in their entirety). The impact of honokiol on the phosphorylation of S6K and 4E-BP1 was therefore examined. As shown in FIG. 15A, honokiol suppressed phosphorylation of both S6K and 4E-BP1. These data further indicate that honokiol suppresses PLD activity in MDA-MB-231 cells and moreover suppresses the phosphorylation of two mTOR substrates induced by PLD activity that correlate with survival signals in cancer cells.

It was recently reported that PLD activity was required for the expression of HIF2α in the renal cell carcinoma cell line 786-0 cells. The effect of honokiol on HIF2α expression was examined in these cells. As reported previously, 1-butanol, which prevents the production of the PLD metabolite phosphatidic acid, suppressed HIF2α expression in these cells (FIG. 15B), confirming the dependence of HIF2α expression on PLD. Also shown in FIG. 15B is that honokiol suppresses HIF2a expression. The data in FIG. 15 demonstrate that honokiol suppresses two downstream effects of PLD—the activation of mTOR and HIF2α expression.

Honokiol Induces Apoptosis in MDA-MB-231 Cells Deprived of Serum

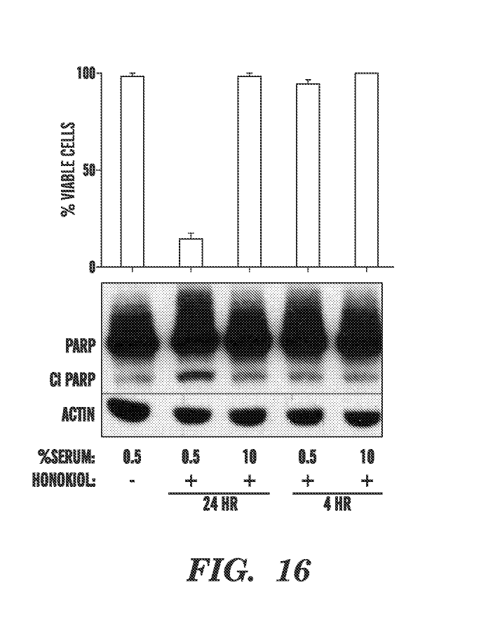

It was previously reported that suppression of PLD activity under conditions of serum withdrawal resulted in apoptosis (Chen et al., “Alternative Phospholipase D/mTOR Survival Signal in Human Breast Cancer Cells,” Oncogene 24:672-9 (2005); Zhong et al., “Phospholipase D Prevents Apoptosis In v-Src-Transformed Rat Fibroblasts and MDA-MB-231 Breast Cancer Cells,” Biochem. Biophys. Res. Comm. 302:615-9 (2003), which is hereby incorporated by reference in its entirety). The effect of honokiol on cell viability and cleavage of the caspase 3 substrate poly-(ADP-ribose) polymerase (“PARP”)—an indicator of apoptotic cell death—was examined in the MDA-MB-231 and 786-0 cells. The effect of honokiol on cell viability and PARP cleavage in 0.5% serum was examined after 4 hr and 24 hr honokiol treatment and in 10% serum after 24 hr honokiol treatment in the MDA-MB-231 cells. As shown in FIG. 16, after 24 hr of honokiol treatment, cell viability was reduced and PARP cleavage increased in 0.5% serum, but not in 10% serum. Importantly, the cells were still viable after 4 hr of honokiol treatment—the time point used in FIGS. 12 and 13 where the effect of honokiol upon PLD activity and on S6K and 4E-BP1 phosphorylation was examined. Honokiol also induced apoptosis in the 786-0 cells, where it has been demonstrated that PLD and HIF2α are critical for survival (Kondo et al, “Inhibition of HIF2α is Sufficient to Suppress pVHL-defective Tumor Growth,” PLoS Biol. 1:439-444 (2003); Kondo et al, “Inhibition of HIF is Necessary for Tumor Suppression by the Von Hippel-Lindau Protein,” Cancer Cell 1:237-246 (2002), which are hereby incorporated by reference in their entirety). These data indicate that honokiol can be used to suppress survival in these two cancer cell lines that rely on PLD activity for their survival under reduced serum conditions.

Honokiol Suppresses Stress-Induced PLD Activity and Induces Apoptosis in T24 Bladder and Calu1 Lung Cancer Cells

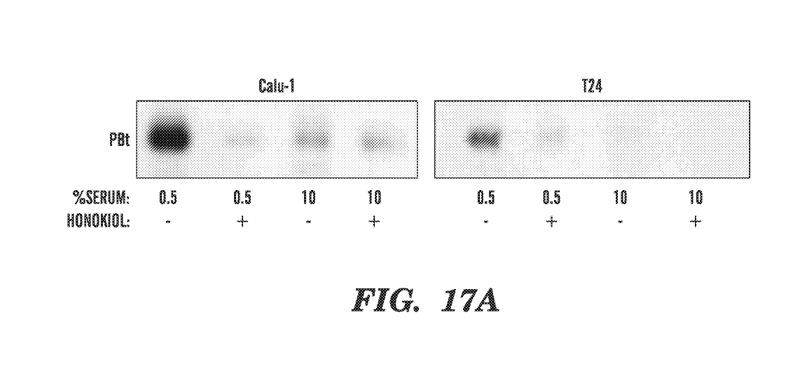

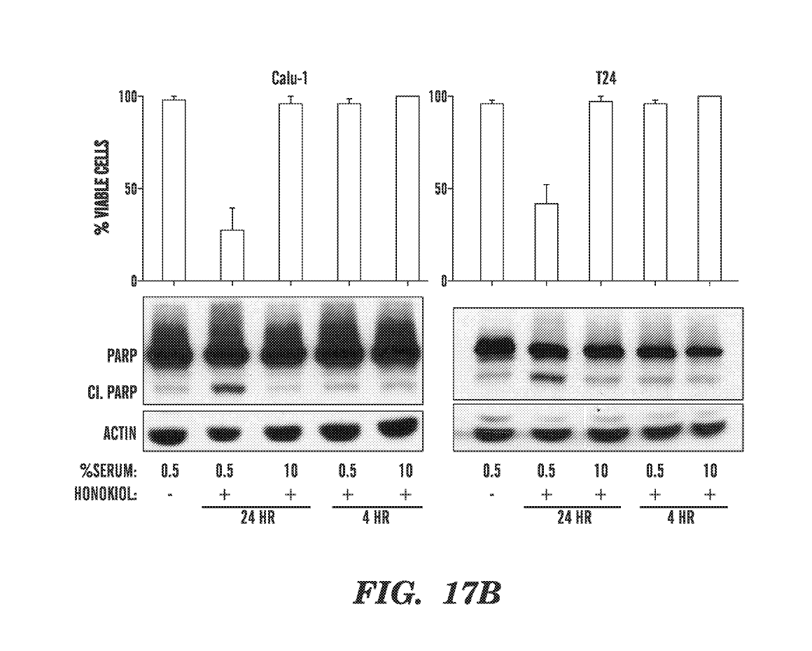

PLD activity is elevated in T24 bladder and Calu-1 lung cancer cells, and the PLD activity in these cells is elevated in response to serum withdrawal (Zheng et al., “Phospholipase D Couples Survival and Migration Signals in Response to Stress in Human Breast Cancer Cells,” J. Biol. Chem. 281:15862-8 (2006); Shi et al., “Elevated Phospholipase D Activity in Human Cancer Cells with Activating Ras Mutations Provides Survival Signal,” Cancer Lett. 258:268-75 (2007), which are hereby incorporated by reference in their entirety). T24 cells have an activated H-Ras mutant (Santos et al., “T24 Human Bladder Carcinoma Oncogene is an Activated Form of the Normal Human Homologue of BALB- and Harvey-MSV Transforming Genes,” Nature 298:343-7 (1982); Taparowsky et al., “Activation of the T24 Bladder Carcinoma Transforming Gene is Linked to a Single Amino Acid Change,” Nature 300:762-5 (1982), which is hereby incorporated by reference in its entirety) and Calu-1 cells an activated K-Ras mutant (Shimizu et al., “Structure of the Ki-ras Gene of the Human Lung Carcinoma Cell Line Calu-1,” Nature 304:497-500 (1983), which is hereby incorporated by reference in its entirety). The PLD activity in both of these cell lines is dependent on Ras (Shi et al., “Elevated Phospholipase D Activity in Human Cancer Cells with Activating Ras Mutations Provides Survival Signal,” Cancer Lett. 258:268-75 (2007), which is hereby incorporated by reference in its entirety). The effect of honokiol on the PLD activity and survival of these human cancer cell lines was examined. As shown in FIG. 17A, honokiol suppressed the stress-induced PLD activity, while having little effect on the PLD activity observed in the presence of serum in both T24 and Calu-1 cells. It was reported previously that the PLD activity in these cells was required for the survival of these cells in the absence of serum (Shi et al., “Elevated Phospholipase D Activity in Human Cancer Cells with Activating Ras Mutations Provides Survival Signal,” Cancer Lett. 258:268-75 (2007), which is hereby incorporated by reference in its entirety). It was therefore examined whether honokiol would induce apoptosis in T24 and Calu-1 cells subjected to serum withdrawal. As shown in FIG. 17B, honokiol induced cell death and PARP cleavage in these cells when these cells were placed in 0.5% serum. These data indicate that the effects of honokiol observed in MDA-MB-231 and 786-0 cells could also be observed in other cancer cells where PLD has been implicated in survival signals.

This example provides evidence that honokiol, a natural product isolated from Magnolia grandiflora, suppresses PLD survival signals in human cancer cells. The PLD activity in MDA-MB-231 cells examined was dependent upon Ras, and honokiol also suppressed Ras activation in these cells. Honokiol was especially effective in cells where Sos was ectopically expressed, indicating that honokiol is suppressing Ras activation by Sos. Importantly, honokiol suppressed the PLD activity that is elevated in response to stress in MDA-MB-231, Calu1, and T24 cancer cells. It is this PLD activity that is likely the PLD activity critical for the survival of the cancer cells under the poorly vascularized conditions of an emerging tumor. These data indicate that honokiol could be a valuable reagent to target an apparent large number of human cancers that depend upon PLD activity and Ras for their survival.

The PLD activity elevated in response to stress in the MDA-MB-231 cells was dependent on Ras in the MDA-MB-231, and also in the Calu1, and T24 cancer cells (Shi et al., “Elevated Phospholipase D Activity in Human Cancer Cells with Activating Ras Mutations Provides Survival Signal,” Cancer Lett. 258:268-75 (2007), which is hereby incorporated by reference in its entirety). As shown in FIG. 12, there was an increase in Ras activation in the MDA-MB-231 cells. This is interesting in that there is an activated K-Ras gene in the MDA-MB-231 cells. Similarly, there are activating mutations to Ras genes in both the Calu1 and T24 cells. It was recently reported that the PLD activity in the T24 cells, where H-Ras is activated, is dependent on the expression of H-Ras; and the PLD activity in the Calu-1 cells, where K-Ras is activated, is dependent on the expression of K-Ras (Shi et al., “Elevated Phospholipase D Activity in Human Cancer Cells with Activating Ras Mutations Provides Survival Signal,” Cancer Lett. 258:268-75 (2007), which is hereby incorporated by reference in its entirety). This suggests that there is increased GTP binding on the mutated Ras genes upon serum withdrawal. However, it is also possible that the increased GTP bound Ras represents activation of the non-mutated Ras—either the Ras protein encoded by the non-mutated wild type allele or other Ras isoforms. In the case of the MDA-MD-231 that would mean that H-Ras was being activated or the wild type K-Ras encoded by the non-mutant allele. Which Ras isoforms were being activated using isoform-specific antibodies to identify the Ras in the pull-down assays was not able to be distinguished due to the lack of specificity of the antibodies. Honokiol blocks PLD activity in the 786-O cells where there is no activated Ras, indicating that honokiol can suppress PLD activity in cells where there is no activated Ras. Thus, while it is not yet clear which Ras isoforms are being activated in response to the stress of serum withdrawal, it is possible that the activation of wild type Ras isoforms could play an important role in the survival of human cancer cells, at least in part by activating PLD.

Previous studies have suggested that honokiol can stimulate apoptosis by through modulation of nuclear factor-KB (NF-KB) activation pathway (Ahn et al., “Honokiol Potentiates Apoptosis, Suppresses Osteoclastogenesis, and Inhibits Invasion through Modulation of Nuclear Factor-KB Activation Pathway,” Mol. Cancer. Res. 4:621-33 (2006); Lee et al., “Growth Inhibitory Effects of Obovatol through Induction of Apoptotic Cell Death in Prostate and Colon Cancer by Blocking of NF-κB,” Eur. J. Pharmacol. 582:17-25 (2008); Tse et al., “Honokiol Inhibits TNF-Alpha-stimulated NF-κB Activation and NF-κB-regulated Gene Expression through Suppression of IKK Activation,” Biochem. Pharmacol. 70:1443-57 (2005), which is hereby incorporated by reference in its entirety). Interestingly, NFκB has been shown to be downstream of both Ral and RalA (Henry et al., “Ral GTPases Contribute to Regulation of Cyclin D1 through Activation of NF-κB,” Mol. Cell. Biol. 20:8084-92 (2000); Fan et al., “Ras Effector Pathways Modulate Scatter Factor-stimulated NF-κB Signaling and Protection Against DNA Damage,” Oncogene 26:4774-96 (2007), which is hereby incorporated by reference in its entirety). Thus, it is possible that both PLD and NFκB are critical targets of honokiol action. It is not known whether there is any dependence of NFκB action on PLD activity, but the data reported here are consistent with the previous studies identifying NFκB as a target of honokiol in that there are common upstream elements that are targeted by honokiol—that being Ras activation.

Honokiol has been shown previously to suppress tumor growth in mouse xenograft studies (Bai et al., “Honokiol, a Small Molecular Weight Natural Product, Inhibits Angiogenesis In Vitro and Tumor Growth In Vivo,” J. Biol. Chem. 278:35501-7 (2003); Wolf et al., “Honokiol, a Natural Biphenyl, Inhibits In Vitro and In Vivo Growth of Breast Cancer through Induction of Apoptosis and Cell Cycle Arrest,” Int. J. Oncol. 30:1529-37 (2007); Ahn et al., “Honokiol Potentiates Apoptosis, Suppresses Osteoclastogenesis, and Inhibits Invasion through Modulation of Nuclear Factor-κB Activation Pathway,” Mol. Cancer. Res. 4:621-33 (2006), which are hereby incorporated by reference in their entirety). Recently, honokiol was shown to suppress the growth of MDA-MB-231 cells in a mouse xenograft study (Wolf et al., “Honokiol, a Natural Biphenyl, Inhibits In Vitro and In Vivo Growth of Breast Cancer through Induction of Apoptosis and Cell Cycle Arrest,” Int. J. Oncol. 30:1529-37 (2007), which is hereby incorporated by reference in its entirety). Importantly, the levels of honokiol employed in the mouse xenograft studies was much higher than previously reported to get levels of plasma honokiol sufficient to suppress PLD activity in this study (Tsai et al., “Pharmacokinetics of Honokiol after Intravenous Administration in Rats Assessed Using High-performance Liquid Chromatography,” J. Chromatogr. B. Biomed. Appl. 655:41-5 (1994), which is hereby incorporated by reference in its entirety). The observation that tumor growth is arrested, but not regressed, suggests that honokiol is not killing all of the cancer cells. As reported here, honokiol was more effective at killing cells when they were in low serum. Emerging tumors are poorly vascularized (Gatenby et al., “Why Do Cancers have High Aerobic Glycolysis?” Nat. Rev. Cancer 4:891-9 (2004), which is hereby incorporated by reference in its entirety) and, therefore, with less exposure to serum growth factors and perhaps other factors in serum, cancer cells in a solid tumor mass could be more susceptible to the apoptotic effects of honokiol. Since honokiol is apparently more effective in stressed cells, it might be possible to make honokiol more effective in vivo with combination therapies that target vascularization. The findings here indicate that honokiol has strong potential as an anti-cancer agent because it targets survival signal in cancer cells and has the potential to resurrect default apoptotic programs. Importantly, early studies with mouse models indicate that honokiol is well tolerated at high concentrations (Bai et al., “Honokiol, a Small Molecular Weight Natural Product, Inhibits Angiogenesis In Vitro and Tumor Growth In Vivo,” J. Biol. Chem. 278:35501-7 (2003); Shigemura et al., “Honokiol, a Natural Plant Product, Inhibits the Bone Metastatic Growth of Human Prostate Cancer Cells,” Cancer 109:1279-89 (2007); Ahn et al., “Honokiol Potentiates Apoptosis, Suppresses Osteoclastogenesis, and Inhibits Invasion through Modulation of Nuclear Factor-KB Activation Pathway,” Mol. Cancer. Res. 4:621-33 (2006); Tsai et al., “Pharmacokinetics of Honokiol After Intravenous Administration in Rats Assessed Using High-performance Liquid Chromatography,” J. Chromatogr. B. Biomed. Appl. 655:41-5 (1994), which are hereby incorporated by reference in their entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of identifying compounds potentially effective in treating a condition mediated by Ras, said method comprising:
providing one or more candidate compounds;
contacting each of the candidate compounds with a cell;
evaluating the effect of the candidate compounds on binding of Son of sevenless to phosphatidic acid; and
identifying candidate compounds which modulate binding of Son of sevenless to phosphatidic acid as compounds potentially effective in treating a condition mediated by Ras.

2. The method of claim 1, wherein candidate compounds which inhibit enzyme binding of Son of sevenless to phosphatidic acid are identified.

3. The method of claim 2, wherein candidate compounds which bind to enzyme phospholipase D2 are identified.

4. The method of claim 1, wherein candidate compounds which bind to Son of sevenless are identified.

5. The method of claim 4, wherein candidate compounds which bind to Son of sevenless at histidine 475 and/or arginine 479 are identified.

6. The method of claim 1, wherein candidate compounds which activate binding of Son of sevenless to phosphatidic acid are identified.
